SEQUENCE LISTING (48 sequences)

SEQ ID NO: 1 - protein, 10 aa, synthetic construct
  REGION 1..10: note = CDRH1
DDSMGSSTYY

SEQ ID NO: 2 - protein, 7 aa, synthetic construct
  REGION 1..7: note = CDRH2
VYYNGVT

SEQ ID NO: 3 - protein, 10 aa, synthetic construct
  REGION 1..10: note = CDRH3
ARQADNWFDP

SEQ ID NO: 4 - protein, 6 aa, synthetic construct
  REGION 1..6: note = CDRL1
QSVGSD

SEQ ID NO: 5 - skipped

SEQ ID NO: 6 - protein, 9 aa, synthetic construct
  REGION 1..9: note = CDRL2 long
LIYGASYRA

SEQ ID NO: 7 - protein, 10 aa, synthetic construct
  REGION 1..10: note = CDRL3
QQYYNWPPYT

SEQ ID NO: 8 - protein, 118 aa, synthetic construct
  REGION 1..118: note = VH
QLQLQESGPGLVRPSETLSLTCSVSDDSMGSSTYYWAWIRQPPGKGLEWIGNVYYNGVTYYNSSLESRLTLSIDPSKNQFSLILSSVTAADTAVYYCARQADNWFDPWGQGTLVTVSS

SEQ ID NO: 9 - protein, 108 aa, synthetic construct
  REGION 1..108: note = VL
EIMMTQSPATLSVSPGERATLSCRASQSVGSDLAWYQQKPGQAPRLLIYGASYRATGIPARFSGGGSGTEFTLTISSMQSEDFAVYYCQQYYNWPPYTFGQGTKVEIK

SEQ ID NO: 10 - protein, 221 aa, synthetic construct
  REGION 1..221: note = Fab heavy chain
QLQLQESGPGLVRPSETLSLTCSVSDDSMGSSTYYWAWIRQPPGKGLEWIGNVYYNGVTYYNSSLESRLTLSIDPSKNQFSLILSSVTAADTAVYYCARQADNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 11 - protein, 215 aa, synthetic construct
  REGION 1..215: note = Fab light chain
EIMMTQSPATLSVSPGERATLSCRASQSVGSDLAWYQQKPGQAPRLLIYGASYRATGIPARFSGGGSGTEFTLTISSMQSEDFAVYYCQQYYNWPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 8 aa, synthetic construct
  REGION 1..8: note = CDRH1
GFTLNNYY

SEQ ID NO: 13 - protein, 8 aa, synthetic construct
  REGION 1..8: note = CDRH2
FNPSSGTR

SEQ ID NO: 14 - protein, 18 aa, synthetic construct
  REGION 1..18: note = CDRH3
ARIGGSTYGRLMTYYFDH

SEQ ID NO: 15 - protein, 6 aa, synthetic construct
  REGION 1..6: note = CDRL1
RSISTT

SEQ ID NO: 16 - skipped

SEQ ID NO: 17 - protein, 9 aa, synthetic construct
  REGION 1..9: note = CDRL2 long
LIYGASTRA

SEQ ID NO: 18 - protein, 9 aa, synthetic construct
  REGION 1..9: note = CDRL3
QQYNDWPVT

SEQ ID NO: 19 - protein, 125 aa, synthetic construct
  REGION 1..125: note = VH
QVQLMQSGAEVQKPGASVKVSCQASGFTLNNYYIHWLRQAPGQGFEWMGIFNPSSGTRTYAQKFQGRISMTADASTTTLYMELSGLTSEDAGVYFCARIGGSTYGRLMTYYFDHWGQGTVVAVSS

SEQ ID NO: 20 - protein, 107 aa, synthetic construct
  REGION 1..107: note = VL
EVVLTQSPVTLSVSPGGRGTLSCRASRSISTTLAWYQQKPGQAPRLLIYGASTRATGIPARFTGSGSGTEFTLTISSLQSEDFAVYYCQQYNDWPVTFGQGTQVEVK

SEQ ID NO: 21 - protein, 228 aa, synthetic construct
  REGION 1..228: note = Fab heavy chain
QVQLMQSGAEVQKPGASVKVSCQASGFTLNNYYIHWLRQAPGQGFEWMGIFNPSSGTRTYAQKFQGRISMTADASTTTLYMELSGLTSEDAGVYFCARIGGSTYGRLMTYYFDHWGQGTVVAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 22 - protein, 214 aa, synthetic construct
  REGION 1..214: note = Fab light chain
EVVLTQSPVTLSVSPGGRGTLSCRASRSISTTLAWYQQKPGQAPRLLIYGASTRATGIPARFTGSGSGTEFTLTISSLQSEDFAVYYCQQYNDWPVTFGQGTQVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 330 aa, synthetic construct
  REGION 1..330: note = IgG heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 24 - protein, 106 aa, synthetic construct
  REGION 1..106: note = IgG lambda light chain
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 107 aa, synthetic construct [sequence omitted]

SEQ ID NO: 26 - protein, 103 aa, synthetic construct
  REGION 1..103: note = Fab heavy chain constant regions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 27 - DNA, 30 bp, synthetic construct
  misc_feature 1..30: note = CDRH1
  source 1..30: mol_type = other DNA
gatgactccatgggcagcagtacttactac

SEQ ID NO: 28 - DNA, 21 bp, synthetic construct
  misc_feature 1..21: note = CDRH2
  source 1..21: mol_type = other DNA
gtctattataatggagtcacc

SEQ ID NO: 29 - DNA, 30 bp, synthetic construct
  misc_feature 1..30: note = CDRH3
  source 1..30: mol_type = other DNA
gcgagacaagcagacaactggttcgacccc

SEQ ID NO: 30 - DNA, 18 bp, synthetic construct
  misc_feature 1..18: note = CDRL1
  source 1..18: mol_type = other DNA
cagagtgttggcagcgac

SEQ ID NO: 31 - skipped

SEQ ID NO: 32 - DNA, 27 bp, synthetic construct
  misc_feature 1..27: note = CDRL2 long
  source 1..27: mol_type = other DNA
ctcatctatggagcatcctacagggcc

SEQ ID NO: 33 - DNA, 30 bp, synthetic construct
  misc_feature 1..30: note = CDRL3
  source 1..30: mol_type = other DNA
cagcagtattacaactggcccccgtacact

SEQ ID NO: 34 - DNA, 354 bp, synthetic construct
  misc_feature 1..354: note = VH
  source 1..354: mol_type = other DNA
cagctgcagctgcaggagtcgggcccaggactggtgaggccctcggagaccctgtccctcacctgcagtgtctctgatgactccatgggcagcagtacttactactgggcctggatccgccagcccccagggaagggactggagtggattgggaatgtctattataatggagtcacctactacaactcgtccctcgagagtcgactcaccttatccatcgacccgtctaagaaccagttctccctgatcttgagttctgtgaccgccgcagacacggccgtatattactgtgcgagacaagcagacaactggttcgacccctggggccagggaaccctggtcaccgtctcgtca

SEQ ID NO: 35 - DNA, 324 bp, synthetic construct
  misc_feature 1..324: note = VL
  source 1..324: mol_type = other DNA
gaaataatgatgacgcagtctccagccaccctgtctgtgtctccaggggaaagagccaccctctcctgcagggccagtcagagtgttggcagcgacttggcttggtaccaacagaaacctggccaggctcccaggctcctcatctatggagcatcctacagggccactggtatcccagccaggttcagtggcggtgggtctgggacagagttcactctcaccatcagcagtatgcagtctgaagattttgcagtttattactgtcagcagtattacaactggcccccgtacacttttggccaggggaccaaggtggagatcaaa

SEQ ID NO: 36 - DNA, 666 bp, synthetic construct
  misc_feature 1..666: note = Fab heavy chain
  source 1..666: mol_type = other DNA
cagctgcagctgcaggagtctggaccaggactggtgcggccctccgagaccctgtctctgacatgctctgtgagcgacgattctatgggcagctccacctactattgggcctggatcagacagccccctggcaagggactggagtggatcggcaacgtgtactataatggcgtgacctactataactctagcctggagtctaggctgacactgagcatcgacccttccaagaatcagttcagcctgatcctgtcctctgtgaccgccgccgatacagccgtgtactattgtgcccggcaggccgacaactggtttgatccttggggacagggcaccctggtgacagtgagctccgcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgttag

SEQ ID NO: 37 - DNA, 648 bp, synthetic construct
  misc_feature 1..648: note = Fab light chain
  source 1..648: mol_type = other DNA
gaaataatgatgacgcagtctccagccaccctgtctgtgtctccaggggaaagagccaccctctcctgcagggccagtcagagtgttggcagcgacttggcttggtaccaacagaaacctggccaggctcccaggctcctcatctatggagcatcctacagggccactggtatcccagccaggttcagtggcggtgggtctgggacagagttcactctcaccatcagcagtatgcagtctgaagattttgcagtttattactgtcagcagtattacaactggcccccgtacacttttggccaggggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 38 - DNA, 24 bp, synthetic construct
  misc_feature 1..24: note = CDRH1
  source 1..24: mol_type = other DNA
ggattcaccctcaacaattactat

SEQ ID NO: 39 - DNA, 24 bp, synthetic construct
  misc_feature 1..24: note = CDRH2
  source 1..24: mol_type = other DNA
ttcaaccctagtagtgggaccaga

SEQ ID NO: 40 - DNA, 54 bp, synthetic construct
  misc_feature 1..54: note = CDRH3
  source 1..54: mol_type = other DNA
gcgagaattggaggctccacttatggccgactaatgacctactactttgatcat

SEQ ID NO: 41 - DNA, 18 bp, synthetic construct
  misc_feature 1..18: note = CDRL1
  source 1..18: mol_type = other DNA
cggagtattagcaccacc

SEQ ID NO: 42 - skipped

SEQ ID NO: 43 - DNA, 27 bp, synthetic construct
  misc_feature 1..27: note = CDRL2 long
  source 1..27: mol_type = other DNA
ctcatttatggtgcttccaccagggcc

SEQ ID NO: 44 - DNA, 27 bp, synthetic construct
  misc_feature 1..27: note = CDRL3
  source 1..27: mol_type = other DNA
cagcagtataatgactggcccgtgacg

SEQ ID NO: 45 - DNA, 375 bp, synthetic construct
  misc_feature 1..375: note = VH
  source 1..375: mol_type = other DNA
caggttcagctaatgcagtctggggctgaggtgcagaagcctggggcctcagtgaaagtgtcctgccaggcatctggattcaccctcaacaattactatattcactggctacgacaggcccctggacaagggtttgagtggatgggaattttcaaccctagtagtgggaccagaacttacgcgcagaagttccagggcagaatcagtatgaccgccgacgcgtcaacgacgacactctacatggagctgagcggcctgacttcggaagacgcgggcgtatatttctgtgcgagaattggaggctccacttatggccgactaatgacctactactttgatcattggggccagggaacggtggtcgccgtctcctca

SEQ ID NO: 46 - DNA, 321 bp, synthetic construct
  misc_feature 1..321: note = VL
  source 1..321: mol_type = other DNA
gaggtagtgttgacacagtctccggtcaccctgtctgtgtctccaggggggagaggcaccctctcctgcagggccagtcggagtattagcaccaccttagcctggtaccagcagaaacctggccaggctcccaggctcctcatttatggtgcttccaccagggccactggtatcccagccaggttcactggcagtgggtctgggacagagttcacgctcaccatcagcagcctacagtctgaagatttcgcagtttattattgtcagcagtataatgactggcccgtgacgttcggccaggggacccaggtggaagtcaag

SEQ ID NO: 47 - DNA, 687 bp, synthetic construct
  misc_feature 1..687: note = Fab heavy chain
  source 1..687: mol_type = other DNA
caggttcagctaatgcagtctggggctgaggtgcagaagcctggggcctcagtgaaagtgtcctgccaggcatctggattcaccctcaacaattactatattcactggctacgacaggcccctggacaagggtttgagtggatgggaattttcaaccctagtagtgggaccagaacttacgcgcagaagttccagggcagaatcagtatgaccgccgacgcgtcaacgacgacactctacatggagctgagcggcctgacttcggaagacgcgggcgtatatttctgtgcgagaattggaggctccacttatggccgactaatgacctactactttgatcattggggccagggaacggtggtcgccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgttag

SEQ ID NO: 48 - DNA, 645 bp, synthetic construct
  misc_feature 1..645: note = Fab light chain
  source 1..645: mol_type = other DNA
gaggtagtgttgacacagtctccggtcaccctgtctgtgtctccaggggggagaggcaccctctcctgcagggccagtcggagtattagcaccaccttagcctggtaccagcagaaacctggccaggctcccaggctcctcatttatggtgcttccaccagggccactggtatcccagccaggttcactggcagtgggtctgggacagagttcacgctcaccatcagcagcctacagtctgaagatttcgcagtttattattgtcagcagtataatgactggcccgtgacgttcggccaggggacccaggtggaagtcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

(19) United States
(12) Patent Application Publication
(10) Pub. No.: US 2025/0129142 A1
(43) Pub. Date: Apr. 24, 2025

(54) ANTIBODIES BINDING TO TETANUS TOXIN AND USES THEREOF

(71) Applicant: INSTITUTE FOR RESEARCH IN BIOMEDICINE, Bellinzona (CH)

(72) Inventor: Antonio LANZAVECCHIA, Porza (CH)

(21) Appl. No.: 18/689,768

(22) PCT Filed: Sep. 6, 2022
(86) PCT No.: PCT/EP2022/074735

§ 371 Date: Mar. 6, 2024

(30) Foreign Application Priority Data
Sep. 7, 2021 (WO) PCT/EP2021/074596

(51) Int. Cl. C07K 16/12; G01N 33/569; G01N 33/68

(52) U.S. Cl. CPC C07K 16/1282; G01N 33/56911; G01N 33/6857; C07K 2317/21; C07K 2317/52; C07K 2317/55; C07K 2317/565; C07K 2317/622; C07K 2317/92; G01N 2333/33

(57) ABSTRACT

The present invention relates to antibodies, and antigen binding fragments thereof, that bind to tetanus toxin. The invention also relates to nucleic acids that encode, and to cells that express such antibodies and antibody fragments. In addition, the invention relates to the use of the antibodies and antibody fragments in the diagnosis, prophylaxis and therapy of infection with Clostridium tetani or tetanus.

The present invention relates to the field of antibodies against tetanus, in particular to antibodies binding to the tetanus toxin. The present invention also relates to the use of such antibodies, e.g. in a method of prophylaxis and treatment of infection with Clostridium tetani or tetanus.

tetanus is caused by infection with the spores of Clostridium tetani. The spores of the bacteria are ubiquitous in the environment, and can be found, for example, in soil, saliva, dust, and manure. They can enter the body through a deep cuts, wounds or burns affecting the nervous system. Most cases occur within fourteen days of infections. Symptoms include jaw cramping or the inability to open the mouth, muscle spasms often in the back, abdomen and extremities, sudden painful muscle spasms often triggered by sudden noises, seizures, headache, fever and sweating. Although tetanus can be prevented by a potent vaccine based on tetanus toxoid, after the age of forty/fifty the titer of antibodies against tetanus toxin decreases. tetanus remains an important public health problem in many parts of the world, but especially in low-income countries or districts, where immunization coverage is low and unclean birth practices are common. WHO estimates that in 2018, 25,000 newborns died from neonatal tetanus.

tetanus is caused by tetanus toxin (also referred to as “tetanus neurotoxin”, TeNT), which is produced by Clostridium tetani in anaerobic conditions. tetanus toxin is an extremely potent neurotoxin having an LD₅₀ of about 2.5-3 ng/kg. The tetanus toxin is encoded in the bacteria by the tetX gene, which is expressed as 150-kDa pre-protein, which is cleaved by either bacterial or host proteases into two parts: a 100 kDa heavy chain and a 50 kDa light chain. The chains are connected by a single disulfide bond. The light chain is a Zn²⁺ metalloprotease, while the heavy chain contains an N-terminal translocation domain (HN) and a C-terminal receptor binding domain (HC).

When patients present necrotic wounds at hospital emergency rooms, the immediate treatment—in addition to aggressive wound care, drugs to control muscle spasms and antibiotics—is usually the administration of polyclonal human antibodies, which are isolated from hyperimmune human donors and known as tetanus immune globulin (TIG). In low-income countries, often hyperimmune horse sera are used, which may generate dangerous hypersensitivity reactions.

However, polyclonal TIG is affected by various drawbacks. It contains only a low percentage of antibodies specifically directed against tetanus toxin, such that usually large amounts of proteins need to injected. This may result in adverse reactions, such as angioedema or anaphylaxis. Moreover, although intrathecal administration of the antibodies appears to be more effective, this approach is limited by the total amount of protein, that can be injected, such that it is not feasible with TIG. Furthermore, the neutralization potency varies from lot to lot of TIG and there is a risk of contamination with unknown viruses or with blood proteins. In addition, TIG contains small amounts of IgA, which may elicit in an immune response in patients with a IgA deficit.

In view of the above, it is the object of the present invention to overcome the drawbacks of the prior art. In particular, it is an object of the present invention to provide antibodies, which specifically bind to tetanus toxin with high binding affinity. Thereby, only small amounts of highly potent antibodies need to be injected.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, for example, x±5%, or x±7%, or x±10%, or x±12%, or x±15%, or x±20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. In some embodiments, the patient is a human.

Doses are often expressed in relation to the bodyweight. Thus, a dose which is expressed as [g, mg, or other unit]/kg (or g, mg etc.) usually refers to [g, mg, or other unit]“per kg (or g, mg etc.) bodyweight”, even if the term “bodyweight” is not explicitly mentioned.

The term “binding” and similar reference usually means “specifically binding”, which does not encompass non-specific sticking.

As used herein, the term “antibody” encompasses various forms of antibodies including, without being limited to, whole antibodies, antibody fragments (such as antigen binding fragments), human antibodies, chimeric antibodies, humanized antibodies, recombinant antibodies and genetically engineered antibodies (variant or mutant antibodies) as long as the characteristic properties according to the invention are retained. In some embodiments, the antibody is a human antibody. In some embodiments, the antibody is a monoclonal antibody. For example, the antibody is a human monoclonal antibody.

As described above, the term “antibody” generally also includes antibody fragments. Fragments of the antibodies may retain the antigen-binding activity of the antibodies. Such fragments are referred to as “antigen-binding fragments”. Antigen-binding fragments include, but are not limited to, single chain antibodies, Fab, Fab′, F(ab′)2, Fv or scFv. Fragments of the antibodies can be obtained from the antibodies by methods that include digestion with enzymes, such as pepsin or papain, and/or by cleavage of disulfide bonds by chemical reduction. Alternatively, fragments of the antibodies can be obtained by recombinant means, for example by cloning and expressing a part (fragment) of the sequences of the heavy and/or light chain. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may be contained in a variety of structures known to the person skilled in the art. In addition, the sequences of the invention may be a component of multispecific molecules in which the sequences of the invention target the epitopes of the invention and other regions of the molecule bind to other targets. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., f. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). In some embodiments, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. As used herein, the term “variable region” (variable region of a light chain (V_{L}), variable region of a heavy chain (V_{H})) denotes each of the pair of light and heavy chains which is involved directly in binding the antibody to the antigen.

Antibodies of the invention can be of any isotype (e.g., IgA, IgG, IgM i.e. an α, γ or μ heavy chain). For example, the antibody is of the IgG type. Within the IgG isotype, antibodies may be IgG1, IgG2, IgG3 or IgG4 subclass, for example IgG1. Antibodies of the invention may have a κ or a λ light chain. In some embodiments, the antibody is of IgG1 type and has a κ light chain.

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

As used herein, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used alone, or in combination, as prophylactic or therapeutic agents upon appropriate formulation, in association with active vaccination, as a diagnostic tool, or as a production tool as described herein.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Antibodies and Antigen-Binding Fragments Thereof.

In a first aspect the present invention provides an (isolated) antibody, or an antigen-binding fragment thereof, which (specifically) binds to tetanus toxin. The present invention also provides an (isolated) antibody, or an antigen-binding fragment thereof, which (specifically) binds to tetanus toxoid.

tetanus toxin (also referred to as tetanus neurotoxin, TeNT) is generally described as having three domains: the light chain (L domain), as well as the N-terminal (HN domain) and the C-terminal (HC domain) portion of the heavy chain. The C-terminal domain of the heavy chain (HC) is responsible for recognizing specific cell-surface receptors and for presynaptic binding. The C-terminal domain of the heavy chain (HC) contains two subdomains, an N-terminal HC-N and a C-terminal HC-C. The C-terminal subdomain HC-C contains a polysialoganglioside binding site and a nidogen binding site, and the N-terminal subdomain HC-N is linked to the HN domain. The N-terminal domain of the heavy chain (HN) is responsible for delivering the L domain into the cytosol by membrane translocation. When the tetanus toxin reaches the cytosol, the disulfide bond between heavy and light chain is reduced and the L domain (light chain) is released into the cytosol of neurons. The L domain is a metalloprotease that blocks neurotransmitter release from inhibitory interneurons of the spinal cord that control the balanced contraction of efferent motor neurons.

Standard methods to assess binding of the antibody according to the present invention, or the antigen-binding fragment thereof, are known to those skilled in the art and include, for example, ELISA (enzyme-linked immunosorbent assay). Thereby, the relative affinities of antibody binding may be determined by measuring the concentration of the antibody (EC₅₀) required to achieve 50% maximal binding at saturation.

An exemplary standard ELISA may be performed as follows: ELISA plates may be coated with a sufficient amount (e.g., 1 μg/ml) of the protein/complex/particle to which binding of the antibody is to be tested (e.g., tetanus toxin). Plates may then be incubated with the antibody to be tested. After washing, antibody binding can be revealed, e.g. using a labelled antibody recognizing the test antibody, such as goat anti-human IgG coupled to alkaline phosphatase. Plates may then be washed, the required substrate (e.g., p-NPP) may be added and plates may be read, e.g. at 405 nm. The relative affinities of antibody binding may be determined by measuring the concentration of mAb (EC₅₀) required to achieve 50% maximal binding at saturation. The EC₅₀ values may be calculated by interpolation of binding curves fitted with a four-parameter nonlinear regression with a variable slope.

In general, the antibody, or an antigen-binding fragment thereof, according to the present invention, may comprise (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e. heavy and light chain: heavy chain variable region (VH) and light chain variable region (VL)), there are typically six CDRs for each antigen receptor (heavy chain: CDRH1, CDRH2, and CDRH3; light chain: CDRL1, CDRL2, and CDRL3). For example, a classical IgG antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a variable region (or each variable region, respectively) may be composed of four framework regions, separated by three CDR's.

The sequences of the heavy chains and light chains of exemplary antibodies of the invention, comprising three different CDRs on the heavy chain and three different CDRs on the light chain were determined. The position of the CDR amino acids are defined according to the IMGT numbering system (IMGT: http://www.imgt.org/; cf. Lefranc, M.-P. et al. (2009) Nucleic Acids Res. 37, D1006-D1012).

In some embodiments, the antibody, or an antigen-binding fragment thereof, comprises (i) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 17, and SEQ ID NO: 18, respectively.

In some embodiments, the antibody, or an antigen-binding fragment thereof, comprises (i) heavy chain CDR1, CDR2, and CDR3 sequences having at least 80% sequence identity with the amino acid sequences of SEQ ID NO: 1, SEQ ID NO: 2, and SEQ ID NO: 3, respectively, and light chain CDR1, CDR2, and CDR3 sequences having at least 80% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 80% sequence identity with the amino acid sequences of SEQ ID NO: 1, SEQ ID NO: 2, and SEQ ID NO: 3, respectively, and light chain CDR1, CDR2, and CDR3 sequences having at least 80% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 80% sequence identity with the amino acid sequences of SEQ ID NO: 12, SEQ ID NO: 13, and SEQ ID NO: 14, respectively, and light chain CDR1, CDR2, and CDR3 sequences having at least 80% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 80% sequence identity with the amino acid sequences of SEQ ID NO: 12, SEQ ID NO: 13, and SEQ ID NO: 14, respectively, and light chain CDR1, CDR2, and CDR3 sequences having at least 80% sequence identity with the amino acid sequences of SEQ ID NO: 15, SEQ ID NO: 17, and SEQ ID NO: 18, respectively.

As used throughout the present specification, “sequence identity” is usually calculated with regard to the full length of the reference sequence (i.e. the sequence recited in the application). Percentage identity, as referred to herein, can be determined, for example, by methods known in the art, such as BLAST using the default parameters specified by the NCBI (the National Center for Biotechnology Information; http://www.ncbi.nlm.nih.gov/) [Blosum 62 matrix; gap open penalty=11 and gap extension penalty=1].

As used throughout this specification, a “sequence variant” has an altered sequence in which one or more of the amino acids or nucleotides in the reference sequence is/are deleted or substituted, and/or one or more amino acids or nucleotides is/are inserted into the reference sequence. As a result of the alterations, the sequence variant usually has a sequence which is at least 70% identical to the reference sequence. Variant sequences which are at least 70% identical have no more than 30 alterations, i.e. any combination of deletions, insertions or substitutions, per 100 amino acids or nucleotides of the reference sequence. Sequence variants may be at least 70%, 75%, 80%, 85%, 90%, 95%, 97%, 98% or 99% identical to the reference sequence (wherein, usually, the higher the %-identity in the sequence variant to the reference sequence, the more preferred is the sequence variant). In a “sequence variant” the functionality of the reference sequence (e.g., in the present case binding to tetanus toxin) may be maintained.

In general, while it is possible to have non-conservative amino acid substitutions, the substitutions are usually conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, cysteine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

The antibody, or an antigen-binding fragment thereof, of the present invention may comprise (i) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 1, SEQ ID NO: 2, and SEQ ID NO: 3, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 4, SEQ ID NO: 5, and SEQ ID NO: 7, respectively; or (ii) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 1, SEQ ID NO: 2, and SEQ ID NO: 3, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 4, SEQ ID NO: 6, and SEQ ID NO: 7, respectively; or (iii) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 12, SEQ ID NO: 13, and SEQ ID NO: 14, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 15, SEQ ID NO: 16, and SEQ ID NO: 18, respectively; or (iv) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 12, SEQ ID NO: 13, and SEQ ID NO: 14, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 15, SEQ ID NO: 17, and SEQ ID NO: 18, respectively.

Accordingly, the antibody or the antigen-binding fragment thereof preferably comprises:
a heavy chain CDR1 sequence according to SEQ ID NO: 1;
a heavy chain CDR2 sequence according to SEQ ID NO: 2;
a heavy chain CDR3 sequence according to SEQ ID NO: 3;
a light chain CDR1 sequence according to SEQ ID NO: 4;
a light chain CDR2 sequence according to SEQ ID NO: 5 or 6; and
a light chain CDR3 sequence according to SEQ ID NO: 7.

Alternatively, the antibody or the antigen-binding fragment thereof preferably comprises:
a heavy chain CDR1 sequence according to SEQ ID NO: 12;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

In some embodiments, the antibody of the invention, or the antigen-binding fragment thereof, comprises (i) a heavy chain variable region (VH) comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8 and a light chain variable region (VL) comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 19 and a light chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain variable region comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8 and a light chain variable region comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 19 and a light chain variable region comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain variable region comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8 and a light chain variable region comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 19 and a light chain variable region comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain variable region comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 8 and a light chain variable region comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 19 and a light chain variable region comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain variable region comprising an amino acid sequence having 90% or more (e.g., 91%, 92%, 93%, 94%, 95%, 96%, 97%, 98%, 99% or more) identity to SEQ ID NO: 8 and a light chain variable region comprising an amino acid sequence having 90% or more (e.g., 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 90% or more (e.g., 91%, 92%, 93%, 94%, 95%, 96%, 97%, 98%, 99% or more) identity to SEQ ID NO: 19 and a light chain variable region comprising an amino acid sequence having 90% or more (e.g., 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain variable region comprising an amino acid sequence having 95% or more (e.g., 96%, 97%, 98%, 99% or more) identity to SEQ ID NO: 8 and a light chain variable region comprising an amino acid sequence having 95% or more (e.g., 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 95% or more (e.g., 96%, 97%, 98%, 99% or more) identity to SEQ ID NO: 19 and a light chain variable region comprising an amino acid sequence having 95% or more (e.g., 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

More specifically, the antibody, or an antigen-binding fragment thereof, preferably comprises a heavy chain variable region comprising or consisting of an amino acid sequence as set forth in SEQ ID NO: 8 and a light chain variable region comprising or consisting of an amino acid sequence as set forth in SEQ ID NO: 9.

Alternatively, the antibody, or an antigen-binding fragment thereof, preferably comprises a heavy chain variable region comprising or consisting of an amino acid sequence as set forth in SEQ ID NO: 19 and a light chain variable region comprising or consisting of an amino acid sequence as set forth in SEQ ID NO: 20.

In some embodiments, the antibody of the invention, or the antigen-binding fragment thereof, comprises (i) a heavy chain comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 10 and a light chain comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 11. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 21 and a light chain comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 22. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 10 and a light chain comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 11. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 21 and a light chain comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 22. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 10 and a light chain comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 11. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 21 and a light chain comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 22. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 10 and a light chain comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 11. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 21 and a light chain comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 22. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain comprising an amino acid sequence having 90% or more (e.g., 91%, 92%, 93%, 94%, 95%, 96%, 97%, 98%, 99% or more) identity to SEQ ID NO: 10 and a light chain comprising an amino acid sequence having 90% or more (e.g., 91%, 92%, 93%, 94%, 95%, 96%, 97%, 98%, 99% or more) identity to SEQ ID NO: 11. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain comprising an amino acid sequence having 90% or more (e.g., 91%, 92%, 93%, 94%, 95%, 96%, 97%, 98%, 99% or more) identity to SEQ ID NO: 21 and a light chain comprising an amino acid sequence having 90% or more (e.g., 91%, 92%, 93%, 94%, 95%, 96%, 97%, 98%, 99% or more) identity to SEQ ID NO: 22. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

In some embodiments, the antibody of the invention, or the antigen-binding fragment thereof, comprises (i) a heavy chain comprising an amino acid sequence having 95% or more (e.g., 96%, 97%, 98%, 99% or more) identity to SEQ ID NO: 10 and a light chain comprising an amino acid sequence having 95% or more (e.g., 96%, 97%, 98%, 99% or more) identity to SEQ ID NO: 11. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain comprising an amino acid sequence having 95% or more (e.g., 96%, 97%, 98%, 99% or more) identity to SEQ ID NO: 21 and a light chain comprising an amino acid sequence having 95% or more (e.g., 96%, 97%, 98%, 99% or more) identity to SEQ ID NO: 22. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

More specifically, the antibody, or an antigen-binding fragment thereof, preferably comprises a heavy chain comprising or consisting of an amino acid sequence as set forth in SEQ ID NO: 10 and a light chain comprising or consisting of an amino acid sequence as set forth in SEQ ID NO: 11.

Alternatively, the antibody, or an antigen-binding fragment thereof, preferably comprises a heavy chain comprising or consisting of an amino acid sequence as set forth in SEQ ID NO: 21 and a light chain comprising or consisting of an amino acid sequence as set forth in SEQ ID NO: 22.

The CDR and VH/VL sequences as well as Fab sequences of exemplified antibodies of the invention, namely antibodies TT104 and TT110 are shown in Table 1 below.

TABLE 1
CDR, VH/VL and Fab sequences (SEQ ID NOs) of exemplified antibodies of the invention.

| | Heavy chain | | | | | Light chain | | | | |
|---|---|---|---|---|---|---|---|---|---|---|
| Antibody name | CDR1 | CDR2 | CDR3 | VH | Fab | CDR1 | CDR2 | CDR3 | VL | Fab |
| TT104 | 1 | 2 | 3 | 8 | 10 | 4 | 5/6 | 7 | 9 | 11 |
| TT110 | 12 | 13 | 14 | 19 | 21 | 15 | 16/17 | 18 | 20 | 22 |

In some embodiments, the antibody of the invention is a human antibody. In some embodiments, the antibody of the invention is a monoclonal antibody. For example, the antibody of the invention may be a human monoclonal antibody.

In some embodiments, the antibody according to the present invention, or an antigen binding fragment thereof, comprises an Fc moiety. The Fc moiety may be derived from human origin, e.g. from human IgG1, IgG2, IgG3, and/or IgG4, such as human IgG1.

As used herein, the term “Fc moiety” refers to a sequence derived from the portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody.

Each of the amino acid positions within an Fc moiety have been numbered herein according to the art-recognized EU numbering system of Kabat, see e.g., by Kabat et al., in “Sequences of Proteins of Immunological Interest”, U.S. Dept. Health and Human Services, 1983 and 1987. The EU index or EU index as in Kabat or EU numbering refers to the numbering of the EU antibody (Edelman G M, Cunningham B A, Gall W E, Gottlieb P D, Rutishauser U, Waxdal M J. The covalent structure of an entire gammaG immunoglobulin molecule. Proc Natl Acad Sci USA. 1969; 63(1):78-85; Kabat E. A., National Institutes of Health (U.S.) Office of the Director, “Sequences of Proteins of Immunological Interest”, 5^{th} edition, Bethesda, MD: U.S. Dept. of Health and Human Services, Public Health Service, National Institutes of Health, 1991, hereby entirely incorporated by reference).

In some embodiments, in the context of the present invention an Fc moiety comprises at least one of: a hinge (e.g., upper, middle, and/or lower hinge region) domain, a CH2 domain, a CH3 domain, or a variant, portion, or fragment thereof. An Fc moiety may comprise at least a hinge domain, a CH2 domain or a CH3 domain. The Fc moiety may be a complete Fc moiety. The Fc moiety may also comprises one or more amino acid insertions, deletions, or substitutions relative to a naturally-occurring Fc moiety. For example, at least one of a hinge domain, CH2 domain or CH3 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a hinge domain (or portion thereof), (v) a CH2 domain (or portion thereof), or (vi) a CH3 domain or portion thereof.

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include Fc receptor (FcR) binding, antibody half-life modulation, ADCC function, protein A binding, protein G binding, and complement binding. The portions of naturally occurring Fc moieties, which are responsible and/or essential for such functions are well known by those skilled in the art. In some embodiments, the antibody according to the present invention comprises a (complete) Fc moiety/Fc region, wherein the interaction/binding with FcR is not compromised.

In general, binding of the antibody to an Fc receptor may be assessed by various methods known to the skilled person, such as ELISA (Hessell A J, Hangartner L, Hunter M, Havenith C E G, Beurskens F J, Bakker J M, Lanigan C M S, Landucci G, Forthal D N, Parren P W H I, et al.: Fc receptor but not complement binding is important in antibody protection against HIV. Nature 2007, 449:101-104; Grevys A, Bern M, Foss S, Bratlie D B, Moen A, Gunnarsen K S, Aase A, Michaelsen T E, Sandlie I, Andersen J T: Fc Engineering of Human IgG1 for Altered Binding to the Neonatal Fc Receptor Affects Fc Effector Functions. 2015, 194:5497-5508) or flow-cytometry (Perez L G, Costa M R, Todd C A, Haynes B F, Montefiori D C: Utilization of immunoglobulin G Fc receptors by human immunodeficiency virus type 1: a specific role for antibodies against the membrane-proximal external region of gp41. J Virol 2009, 83:7397-7410; Piccoli L, Campo I, Fregni C S, Rodriguez B M F, Minola A, Sallusto F, Luisetti M, Corti D, Lanzavecchia A: Neutralization and clearance of GM-CSF by autoantibodies in pulmonary alveolar proteinosis. Nat Commun 2015, 6:1-9).

For example, FcR binding can be mediated by the interaction of the Fc moiety (of an antibody) with Fc receptors (FcRs), which are specialized cell surface receptors on hematopoietic cells. Fc receptors belong to the immunoglobulin superfamily, and were shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Therefore, the Fc moiety may provide cross-linking of receptors (FcγR). In humans, three classes of FcγR have been characterized, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is known to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homolougous; and (iii) FcγRIII (CD16), which binds IgG with medium to low affinity and exists as two types: FcγRIIIA found on NK cells, macrophages, eosinophils and some monocytes and T cells and mediating ADCC and FcγRIIIB, which is highly expressed on neutrophils. FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, 75% of all FcγRIIB is found in the liver (Ganesan, L. P. et al., 2012: FcγRIIb on liver sinusoidal endothelium clears small immune complexes. Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

Accordingly, antibodies, and antigen binding fragments thereof, of the invention may be able to bind to FcγRIIb, for example antibodies comprising an Fc moiety for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcγRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). Accordingly, the antibodies, or antigen binding fragments thereof, of the invention may comprise an engineered Fc moiety with the mutations S267E and L328F, in particular as described by Chu, S. Y. et al., 2008: Inhibition of B cell receptor-mediated activation of primary human B cells by coengagement of CD19 and FcγRIIb with Fc-engineered antibodies. Molecular Immunology 45, 3926-3933.

On B-cells it seems to function to suppress further immunoglobulin production and isotype switching to say for example the IgE class. On macrophages, FcγRIIB acts to inhibit phagocytosis as mediated through FcγRIIA. On eosinophils and mast cells the b form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into IgG1 and IgG4, reduces binding to FcγRI by 10³-fold and eliminated the human monocyte response to antibody-sensitized red blood cells (Armour, K. L., et al. Eur. J. Immunol 29 (1999) 2613-2624). Regarding FcγRII binding, reduced binding for FcγRIIA is found e.g. for IgG mutation of at least one of E233-G236, P238, D265, N297, A327, P329, D270, Q295, A327, R292 and K414. Regarding FcγRIII binding, reduced binding to FcγRIIIA is found e.g. for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above mentioned mutation sites and methods for measuring binding to FcγRI and FcγRIIA are described in Shields, R. L., et al., J. Biol. Chem. 276 (2001) 6591-6604. For example, a single (S239D or 1332E), double (S239D/1332E), and triple mutations (S239D/1332E/A330L) improved the affinity against human FcγRIIIa. Furthermore, the addition of the mutation G236A to S239D/1332E improved not only FcγRIIa:FcγRIIb ratio, but also enhanced binding to FcγRIIIa. Accordingly, the mutations G236A/S239D/A330L/1332E were described to enhance engagement of FcγRIIa and FcγRIIIa.

Regarding binding to the crucial FcγRII, two regions of native IgG Fc appear to be critical for interactions of FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

For example, the Fc moiety may comprise or consist of at least the portion of an Fc moiety that is known in the art to be required for FcRn binding or extended half-life. Alternatively or additionally, the Fc moiety of the antibody of the invention comprises at least the portion of known in the art to be required for Protein A binding and/or the Fc moiety of the antibody of the invention comprises at least the portion of an Fc molecule known in the art to be required for protein G binding. The Fc moiety may comprise at least the portion known in the art to be required for FcγR binding. As outlined above, an Fc moiety may thus at least comprise (i) the lower hinge site of native IgG Fc, in particular amino acid residues L, L, G, G (234-237, EU numbering), and (ii) the adjacent region of the CH2 domain of native IgG Fc, in particular a loop and strands in the upper CH2 domain adjacent to the lower hinge region, e.g. in a region of P331, for example a region of at least 3, 4, 5, 6, 7, 8, 9, or 10 consecutive amino acids in the upper CH2 domain of native IgG Fc around P331, e.g. between amino acids 320 and 340 (EU numbering) of native IgG Fc.

Furthermore, the antibody according to the present invention can be modified by introducing (random) amino acid mutations into particular region of the CH2 or CH3 domain of the heavy chain in order to alter their binding affinity for FcR and/or their serum half-life in comparison to unmodified antibodies. Examples of such modifications include, but are not limited to, substitutions of at least one amino acid from the heavy chain constant region selected from the group consisting of amino acid residues 250, 314, and 428. Further examples of such Fc modifications are described in Saxena A, Wu D. Advances in Therapeutic Fc Engineering—Modulation of IgG-Associated Effector Functions and Serum Half-life. Front Immunol. 2016; 7:580, which is incorporated herein by reference. In some embodiments, the antibody may comprise the “YTE” mutations (M252Y/S254T/T256E; EU numbering). In some embodiments, the antibody may comprise the mutations M428L and/or N434S in the heavy chain constant region (EU numbering).

In some embodiments, the antibody, or antigen binding fragment thereof, according to the present invention comprises an Fc region. As used herein, the term “Fc region” refers to the portion of an immunoglobulin formed by two or more Fc moieties of antibody heavy chains. For example, the Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2. The Fc region may be dimeric. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties.

The Fc moieties of the Fc region may be of the same or different class and/or subclass. For example, the Fc moieties may be derived from an immunoglobulin (e.g., a human immunoglobulin) of an IgG1, IgG2, IgG3 or IgG4 subclass. The Fc moieties of the Fc region may be of the same class and subclass. However, the Fc region (or one or more Fc moieties of an Fc region) may also be chimeric, whereby a chimeric Fc region may comprise Fc moieties derived from different immunoglobulin classes and/or subclasses. For example, at least two of the Fc moieties of a dimeric or single-chain Fc region may be from different immunoglobulin classes and/or subclasses. Additionally or alternatively, the chimeric Fc regions may comprise one or more chimeric Fc moieties. For example, the chimeric Fc region or moiety may comprise one or more portions derived from an immunoglobulin of a first subclass (e.g., an IgG1, IgG2, or IgG3 subclass) while the remainder of the Fc region or moiety is of a different subclass. For example, an Fc region or moiety of an Fc polypeptide may comprise a CH2 and/or CH3 domain derived from an immunoglobulin of a first subclass (e.g., an IgG1, IgG2 or IgG4 subclass) and a hinge region from an immunoglobulin of a second subclass (e.g., an IgG3 subclass). For example, the Fc region or moiety may comprise a hinge and/or CH2 domain derived from an immunoglobulin of a first subclass (e.g., an IgG4 subclass) and a CH3 domain from an immunoglobulin of a second subclass (e.g., an IgG1, IgG2, or IgG3 subclass). For example, the chimeric Fc region may comprise an Fc moiety (e.g., a complete Fc moiety) from an immunoglobulin for a first subclass (e.g., an IgG4 subclass) and an Fc moiety from an immunoglobulin of a second subclass (e.g., an IgG1, IgG2 or IgG3 subclass). For example, the Fc region or moiety may comprise a CH2 domain from an IgG4 immunoglobulin and a CH3 domain from an IgG1 immunoglobulin. For example, the Fc region or moiety may comprise a CH1 domain and a CH2 domain from an IgG4 molecule and a CH3 domain from an IgG1 molecule. For example, the Fc region or moiety may comprise a portion of a CH2 domain from a particular subclass of antibody, e.g., EU positions 292-340 of a CH2 domain. For example, an Fc region or moiety may comprise amino acids a positions 292-340 of CH2 derived from an IgG4 moiety and the remainder of CH2 derived from an IgG1 moiety (alternatively, 292-340 of CH2 may be derived from an IgG1 moiety and the remainder of CH2 derived from an IgG4 moiety).

Moreover, an Fc region or moiety may (additionally or alternatively) for example comprise a chimeric hinge region. For example, the chimeric hinge may be derived, e.g. in part, from an IgG1, IgG2, or IgG4 molecule (e.g., an upper and lower middle hinge sequence) and, in part, from an IgG3 molecule (e.g., an middle hinge sequence). In another example, an Fc region or moiety may comprise a chimeric hinge derived, in part, from an IgG1 molecule and, in part, from an IgG4 molecule. In another example, the chimeric hinge may comprise upper and lower hinge domains from an IgG4 molecule and a middle hinge domain from an IgG1 molecule. Such a chimeric hinge may be made, for example, by introducing a proline substitution (Ser228Pro) at EU position 228 in the middle hinge domain of an IgG4 hinge region. In other embodiments, the chimeric hinge can comprise amino acids at EU positions 233-236 are from an IgG2 antibody and/or the Ser228Pro mutation, wherein the remaining amino acids of the hinge are from an IgG4 antibody. Further chimeric hinges, which may be used in the Fc moiety of the antibody according to the present invention are described in US 2005/0163783 A1.

In some embodiments, the Fc moiety, or the Fc region, comprises or consists of an amino acid sequence derived from a human immunoglobulin sequence (e.g., from an Fc region or Fc moiety from a human IgG molecule). However, the Fc moiety, or the Fc region, may comprise one or more amino acids from another mammalian species. For example, a primate Fc moiety or a primate binding site may be included in the antibody, or antigen-binding fragment. Alternatively, one or more murine amino acids may be present in the Fc moiety or in the Fc region.

In some embodiments, the antibody, or antigen-binding fragment, comprises, in particular in addition to an Fc moiety as described above, other parts derived from a constant region, in particular from a constant region of IgG, such as a constant region of (human) IgG1. The antibody according to the present invention may comprise, in particular in addition to an Fc moiety as described above, all other parts of the constant regions, in particular all other parts of the constant regions of IgG (such as (human) IgG1). In other words, the antibody, or antigen-binding fragment, may comprise a (complete) Fc region derived from human IgG1. In some embodiments, the antibody, or antigen-binding fragment, comprises, in particular in addition to a (complete) Fc region derived from human IgG1 also all other parts of the constant regions of IgG, such as all other parts of the constant regions of (human) IgG1.

However, in general, antibodies of the invention can be of any isotype (e.g., IgA, IgG, IgM i.e. an α, γ or μ heavy chain). For example, the antibody may be of the IgG type. Within the IgG isotype, antibodies may be IgG1, IgG2, IgG3 or IgG4 subclass, for example IgG1. Antibodies of the invention may have a κ or a λ light chain. In some embodiments, the antibody is of IgG1 type and has a lambda or kappa light chain.

In some embodiments, the antibody is of the human IgG1 type. The antibody may be of any allotype. The term “allotype” refers to the allelic variation found among the IgG subclasses. For example, the antibody may be of the G1 m1 (or G1 m(a)) allotype, of the G1 m2 (or G1 m(x)) allotype, of the G1 m3 (or G1 m(f)) allotype, and/or of the G1 m17 (or Gm(z)) allotype. The G1 m3 and G1 m17 allotypes are located at the same position in the CH1 domain (position 214 according to EU numbering). G1m3 corresponds to R214 (EU), while G1m17 corresponds to K214 (EU). The G1 m1 allotype is located in the CH3 domain (at positions 356 and 358 (EU)) and refers to the replacements E356D and M358L. The G1 m2 allotype refers to a replacement of the alanine in position 431 (EU) by a glycine. The G1 m1 allotype may be combined, for example, with the G1 m3 or the G1m17 allotype. In some embodiments, the antibody is of the allotype G1m3 with no G1m1 (G1m3,-1). In some embodiments, the antibody is of the G1m17,1 allotype. In some embodiments, the antibody is of the G1 m3,1 allotype. In some embodiments, the antibody is of the allotype G1m17 with no G1 m1 (G1m17,-1). Optionally, these allotypes may be combined (or not combined) with the G1 m2, G1 m27 or G1 m28 allotype. For example, the antibody may be of the G1m17,1,2 allotype.

In general, the antibody, or antigen-binding fragment, according to the present invention may be glycosylated. N-linked glycans attached to the CH2 domain of a heavy chain, for instance, can influence C1q and FcR binding, with glycosylated antibodies having lower affinity for these receptors. Accordingly, the CH2 domain of the Fc moiety of the antibody according to the present invention may comprise one or more mutations, in which a glycosylated residue is substituted by a non-glycosylated residue. For example, the antibody's glycans do not lead to a human immunogenic response after administration.

Example sequences of constant regions are the amino acid sequences according to SEQ ID NOs: 23-26. For example, the amino acid sequence of IgG1 CH1-CH2-CH3 is according to SEQ ID NO: 23 or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity. The light chain constant region may be according to SEQ ID NO: 24 or 25; or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity.

As outlined above, the present invention encompasses antigen-binding fragments. An antigen-binding fragment may or may not comprise an Fc moiety, in particular a portion of a complete Fc moiety. In some embodiments, the antibody, or antigen-binding fragment thereof, is selected from Fab, Fab′, F(ab′)2, Fv or scFv. For example, F(ab′)2 (which may be obtained by pepsin cleavage or recombinant expression) as well as Fab′ (which can be obtained from F(ab′)2 or by recombinant expression) usually includes the hinge region.

In some embodiments, the antibody, or antigen-binding fragment, may be a single-chain antibody (or fragment). The single-chain antibody (or fragment) may encode the complete set of six CDRs, i.e. include the three heavy chain CDRs as well as the three light chain CDRs. More specifically, the single-chain antibody (or fragment) may include a heavy chain variable region (VH) as well as a light chain variable region (VL), for example including the VH and VL sequences as described above.

In some embodiments, the antibody, or antigen-binding fragment thereof, does not comprise an Fc moiety. In other words, the antibody, or antigen-binding fragment, may not comprise any of the hinge region, CH2 region and CH3 region. In certain embodiments, the antibody, or antigen-binding fragment thereof, is a Fab. A Fab typically includes, in addition to the heavy and light chain variable domains, a single constant region of the heavy chain (e.g., CH1) and of the light chain (e.g., CL). A Fab may be obtained, for example, by papain cleavage or by recombinant expression using, for example, a stop-codon introduced after the nucleic acid sequence encoding the heavy chain VH and CH1 regions (in particular directly after the CH1 encoding sequence). For example, the amino acid sequence of the heavy chain constant regions of the Fab (CH1 region) may be according to SEQ ID NO: 26, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity. The light chain constant region of the Fab usually corresponds to that of the “complete” immunoglobulin and, thus, may be according to SEQ ID NO: 24 or 25; or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity.

In some embodiments, the Fab comprises or consists of a heavy chain sequence according to SEQ ID NO: 10, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity; and a light chain sequence according to SEQ ID NO: 11, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained.

In other embodiments, the Fab comprises or consists of a heavy chain sequence according to SEQ ID NO: 21, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity; and a light chain sequence according to SEQ ID NO: 22, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

Antibodies of the invention also include hybrid antibody molecules that comprise the six CDRs from an antibody of the invention as defined above and one or more CDRs from another antibody to an antigen. For example, the antibody may be bispecific.

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the invention, or antigen-binding fragments thereof, may be provided in purified form. Typically, the antibody, or antigen-binding fragment, will be present in a composition that is substantially free of other polypeptides e.g., where less than 90% (by weight), usually less than 60% and more usually less than 50% of the composition is made up of other polypeptides.

Antibodies of the invention may be immunogenic in non-human (or heterologous) hosts e.g., in mice. In particular, the antibodies may have an idiotope that is immunogenic in non-human hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

### Nucleic Acids

In another aspect, the invention also provides a nucleic acid molecule comprising a polynucleotide encoding the antibody according to the present invention, or an antigen-binding fragment thereof, as described above.

In some embodiments, the nucleic acid molecule comprises one or more polynucleotide(s) encoding the exemplified antibodies of the invention (e.g., as described in Table 1 above), or a sequence variant thereof as described herein (e.g., having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity as described above).

Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of an antibody. In other words, the light chain and the heavy chain of the antibody may be encoded by the same nucleic acid molecule (e.g., in bicistronic manner). Alternatively, the light chain and the heavy chain of the antibody may be encoded by distinct nucleic acid molecules.

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, a heterologous UTR (e.g., for optimal translation/expression), a heterologous Poly-A-tail, and the like.

A nucleic acid molecule is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, or an antigen-binding fragment thereof, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33 (Web Server issue):W526-31; or, for example, Genscript's OptimumGene™ algorithm (as described in US 2011/0081708 A1).

For example, the nucleic acid molecule of the invention may comprise a nucleic acid sequence as set forth in any one of SEQ ID NOs 27-48; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

Exemplified nucleic acid sequences encoding the CDRs, VH/VL or Fab sequences of exemplified antibodies of the invention are shown in Table 2 below.

TABLE 2
Exemplified nucleic acid CDRs, VH/VL or Fab sequences (SEQ
ID NOs) of exemplified antibodies of the invention.

| | Heavy chain | | | | | Light chain | | | | |
|---|---|---|---|---|---|---|---|---|---|---|
| Antibody name | CDR1 | CDR2 | CDR3 | VH | Fab | CDR1 | CDR2 | CDR3 | VL | Fab |
| TT104 | 27 | 28 | 29 | 34 | 36 | 30 | 31/32 | 33 | 35 | 37 |
| TT110 | 38 | 39 | 40 | 45 | 47 | 41 | 42/43 | 44 | 46 | 48 |

Accordingly, the nucleic acid molecule may comprise:
(i) a polynucleotide according to SEQ ID NO: 27 or 38 (or a sequence variant thereof); a polynucleotide according to SEQ ID NO: 28 or 39 (or a sequence variant thereof); a polynucleotide according to SEQ ID NO: 29 or 40 (or a sequence variant thereof); a polynucleotide according to SEQ ID NO: 30 or 41 (or a sequence variant thereof); a polynucleotide according to (a) SEQ ID NO: 31 or 32 (or a sequence variant thereof) or (b) SEQ ID NO: 42 or 43 (or a sequence variant thereof); and a polynucleotide according to SEQ ID NO: 33 or 44 (or a sequence variant thereof);
(ii) a polynucleotide according to SEQ ID NO: 34 or 45 (or a sequence variant thereof); and a polynucleotide according to SEQ ID NO: 35 or 46 (or a sequence variant thereof); or
(iii) a polynucleotide according to SEQ ID NO: 36 or 47 (or a sequence variant thereof); and a polynucleotide according to SEQ ID NO: 37 or 48 (or a sequence variant thereof).

The present invention also provides a combination of a first and a second nucleic acid molecule, wherein the first nucleic acid molecule comprises a polynucleotide encoding the heavy chain of the antibody, or an antigen-binding fragment thereof, of the present invention; and the second nucleic acid molecule comprises a polynucleotide encoding the corresponding light chain of the same antibody, or the same antigen-binding fragment thereof. The above description regarding the (general) features of the nucleic acid molecule of the invention applies accordingly to the first and second nucleic acid molecule of the combination. Accordingly, one or both of the polynucleotides encoding the heavy and/or light chain(s) of the antibody, or an antigen-binding fragment thereof, may be codon-optimized. For example, the combination may comprise a nucleic acid sequence as set forth in any one of SEQ ID NOs 27-48; or a sequence variant thereof having at least 70%, at least 75%, at least 80%, at least 85%, at least 88%, at least 90%, at least 92%, at least 95%, at least 96%, at least 97%, at least 98% or at least 99% sequence identity. In some embodiments, the combination of a first and a second nucleic acid molecule, wherein the first nucleic acid molecule comprises a polynucleotide encoding the heavy chain of the antibody, or an antigen-binding fragment thereof, of the present invention; and the second nucleic acid molecule comprises a polynucleotide encoding the corresponding light chain of the same antibody, or the same antigen-binding fragment thereof, are as described in the following.

The present invention also provides a combination of a first and a second nucleic acid molecule, wherein
the first nucleic acid molecule comprises a polynucleotide encoding the heavy chain of an antibody, or an antigen-binding fragment thereof, the polynucleotide comprising: (a) nucleotide sequences according to SEQ ID NOs 27, 28 and 29 (or sequence variants thereof); or (b) nucleotide sequences according to SEQ ID NOs 38, 39 and 40 (or sequence variants thereof); and
the second nucleic acid molecule comprises a polynucleotide encoding the light chain of an antibody, or an antigen-binding fragment thereof, the polynucleotide comprising: (c) nucleotide sequences according to SEQ ID NOs 30, 31 (or 32) and 33 (or sequence variants thereof); or (d) nucleotide sequences according to SEQ ID NOs 41, 42 (or 43) and 44 (or sequence variants thereof).

Such a combination usually encodes the antibody, or an antigen-binding fragment thereof, of the present invention as described above. Again, the above description regarding the (general) features of the nucleic acid molecule of the invention applies accordingly to the first and second nucleic acid molecule of the combination.

In some embodiments,
the first nucleic acid molecule comprises a polynucleotide comprising (a) a nucleotide sequence according to SEQ ID NO: 34 (or a sequence variant thereof); or (b) a nucleotide sequence according to SEQ ID NO: 45 (or a sequence variant thereof); and
the second nucleic acid molecule comprises a polynucleotide comprising (c) a nucleotide sequence according to SEQ ID NO: 35 (or a sequence variant thereof); or (d) a nucleotide sequence according to SEQ ID NO: 46 (or a sequence variant thereof).

In some embodiments,
the first nucleic acid molecule comprises a polynucleotide comprising (a) a nucleotide sequence according to SEQ ID NO: 36 (or a sequence variant thereof); or (b) a nucleotide sequence according to SEQ ID NO: 47 (or a sequence variant thereof); and
the second nucleic acid molecule comprises a polynucleotide comprising (c) a nucleotide sequence according to SEQ ID NO: 37 (or a sequence variant thereof); or (d) a nucleotide sequence according to SEQ ID NO: 48 (or a sequence variant thereof).

It is understood for the exemplified sequences as described herein, that such combinations of SEQ ID NOs, which correspond to those of either antibody TT104 or antibody TT110, as shown in Table 2 above, are preferred.

### Vector

Further included within the scope of the invention are vectors, for example, expression vectors, comprising a nucleic acid molecule according to the present invention. Usually, a vector comprises a nucleic acid molecule as described above.

The present invention also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

### Cells

In a further aspect, the present invention also provides cell expressing the antibody according to the present invention, or an antigen-binding fragment thereof; and/or comprising the vector (or the combination of vectors) according the present invention.

Examples of such cells include but are not limited to, eukaryotic cells, e.g., yeast cells, animal cells or plant cells. Other examples of such cells include but are not limited, to prokaryotic cells, e.g. E co/i. In some embodiments, the cells are mammalian cells, such as a mammalian cell line. Examples include human cells, CHO cells, HEK293T cells, PER.C6 cells, NS0 cells, human liver cells, myeloma cells or hybridoma cells.

The cell may be transfected with a vector according to the present invention, for example with an expression vector. The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present invention may be transfected stably or transiently with the vector according to the present invention, e.g. for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen-binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state. Such a PTM may result in a functional difference (e.g., reduced immunogenicity). Accordingly, the antibody of the invention, or the antigen-binding fragment thereof, may have a post-translational modification, which is distinct from the naturally produced antibody (e.g., an antibody of an immune response in a human).

### Production of Antibodies

Antibodies according to the invention can be made by any method known in the art. For example, the general methodology for making monoclonal antibodies using hybridoma technology is well known (Kohler, G. and Milstein, C., 1975; Kozbar et al. 1983). In some embodiments, the alternative EBV immortalization method described in WO2004/076677 is used.

In some embodiments, the method as described in WO 2004/076677, which is incorporated herein by reference, is used. In this method B cells producing the antibody of the invention are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another exemplified method is described in WO 2010/046775. In this method plasma cells are cultured in limited numbers, or as single plasma cells in microwell culture plates. Antibodies can be isolated from the plasma cell cultures. Further, from the plasma cell cultures, RNA can be extracted and PCR can be performed using methods known in the art. The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293T, PER.C6, NS0, myeloma or hybridoma cells. Also, prokaryotic, e.g. bacterial host cell expression systems may be used for the production of antibody molecules, such as complete antibody molecules. Suitable bacterial host cells include, but are not limited to, E co/i cells.

The present invention also provides a process for the production of an antibody molecule according to the present invention comprising culturing a (heterologous) host cell comprising a vector encoding a nucleic acid of the present invention under conditions suitable for expression of protein from DNA encoding the antibody molecule of the present invention, and isolating the antibody molecule.

For production of the antibody comprising both heavy and light chains, a cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Antibodies according to the invention may be produced by (i) expressing a nucleic acid sequence according to the invention in a host cell, e.g. by use of a vector according to the present invention, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells of the invention can be used in various ways e.g., as a source of monoclonal antibodies, as a source of nucleic acid (DNA or mRNA) encoding a monoclonal antibody of interest, for research, etc.

The invention also provides a composition comprising immortalized B memory cells or transfected host cells that produce antibodies according to the present invention.

The immortalized B cell clone or the cultured plasma cells of the invention may also be used as a source of nucleic acid for the cloning of antibody genes for subsequent recombinant expression. Expression from recombinant sources may be more common for pharmaceutical purposes than expression from B cells or hybridomas e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a (heterologous) host cell in order to permit expression of the antibody of interest in that host cell.

Similarly, the invention also provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from an immortalized B cell clone or a cultured plasma cell of the invention. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells or plasma cells are well known in the art (e.g., see Chapter 4 of Kuby Immunology, 4th edition, 2000).

The transfected host cell may be a eukaryotic cell, including yeast and animal cells, particularly mammalian cells (e.g., CHO cells, NS0 cells, human cells such as PER.C6 or HKB-11 cells, myeloma cells, or a human liver cell), as well as plant cells. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The invention also provides a method for preparing one or more nucleic acid molecules (e.g., heavy and light chain genes) that encode an antibody of interest, comprising the steps of: (i) preparing an immortalized B cell clone or culturing plasma cells according to the invention; (ii) obtaining from the B cell clone or the cultured plasma cells nucleic acid that encodes the antibody of interest. Further, the invention provides a method for obtaining a nucleic acid sequence that encodes an antibody of interest, comprising the steps of: (i) preparing an immortalized B cell clone or culturing plasma cells according to the invention; (ii) sequencing nucleic acid from the B cell clone or the cultured plasma cells that encodes the antibody of interest.

The invention further provides a method of preparing nucleic acid molecule(s) that encode an antibody of interest, comprising the step of obtaining the nucleic acid that was obtained from a transformed B cell clone or cultured plasma cells of the invention. Thus the procedures for first obtaining the B cell clone or the cultured plasma cell, and then obtaining nucleic acid(s) from the B cell clone or the cultured plasma cells can be performed at different times by different people in different places (e.g., in different countries).

The invention also comprises a method for preparing an antibody (e.g., for pharmaceutical use) according to the present invention, comprising the steps of: (i) obtaining and/or sequencing one or more nucleic acids (e.g., heavy and light chain genes) from the selected B cell clone or the cultured plasma cells expressing the antibody of interest; (ii) inserting the nucleic acid(s) into or using the nucleic acid(s) sequence(s) to prepare an expression vector; (iii) transfecting a host cell that can express the antibody of interest; (iv) culturing or sub-culturing the transfected host cells under conditions where the antibody of interest is expressed; and, optionally, (v) purifying the antibody of interest.

The invention also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest, wherein said transfected host cell population has been prepared by (i) providing nucleic acid(s) encoding a selected antibody of interest that is produced by a B cell clone or cultured plasma cells prepared as described above, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest. Thus the procedures for first preparing the recombinant host cell and then culturing it to express antibody can be performed at very different times by different people in different places (e.g., in different countries).

### Pharmaceutical Composition

The present invention also provides a pharmaceutical composition comprising one or more of:
(i) the antibody of the present invention, or an antigen-binding fragment thereof;
(ii) the nucleic acid or the combination of nucleic acids of the present invention;
(iii) the vector or the combination of vectors of the present invention; and/or
(iv) the cell expressing the antibody according to the present invention or comprising the vector according to the present invention
and, optionally, a pharmaceutically acceptable excipient, diluent or carrier.

In other words, the present invention also provides a pharmaceutical composition comprising the antibody according to the present invention, the nucleic acid according to the present invention, the vector according to the present invention and/or the cell according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition according to the present invention is not an active component in respect to infection with Clostridium tetani or tetanus.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin®, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody according to the present invention. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions of the invention are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular the antibodies according to the present invention. For example, the vehicle may be a physiologically acceptable liquid, which is suitable for storing, transporting, and/or administering a pharmaceutically active compound, in particular the antibodies according to the present invention. Once formulated, the compositions of the invention can be administered directly to the subject. In some embodiments the compositions are adapted for administration to mammalian, e.g., human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. In some embodiments, the pharmaceutical composition may be administered to the central nervous system. Accordingly, for example it may be administered via the intrathecal, intracerebroventricular, intracerebral, epidural, transnasal, intranasal, or perispinal route of administration. Hyposprays may also be used to administer the pharmaceutical compositions of the invention. Optionally, the pharmaceutical composition may be prepared for oral administration, e.g. as tablets, capsules and the like, for topical administration, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound according to the present invention that is to be given to an individual, administration is usually in an “effective amount”, e.g. in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The inventive pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the inventive transporter cargo conjugate molecule as defined above, is combined with emulsifying and suspending agents. If desired, certain sweetening, flavoring or coloring agents may also be added.

The inventive pharmaceutical composition may also be administered topically, especially when the target of treatment includes areas or organs readily accessible by topical application, e.g. including accessible epithelial tissue. Suitable topical formulations are readily prepared for each of these areas or organs. For topical applications, the inventive pharmaceutical composition may be formulated in a suitable ointment, containing the inventive pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. In particular, the pharmaceutical composition may be provided as single-dose product. In some embodiments, the amount of the antibody in the pharmaceutical composition—in particular if provided as single-dose product—does not exceed 200 mg, for example it does not exceed 100 mg or 50 mg.

For a single dose, e.g. a daily, weekly or monthly dose, the amount of the antibody in the pharmaceutical composition according to the present invention, may not exceed 1 g or 500 mg. In some embodiments, for a single dose, the amount of the antibody in the pharmaceutical composition according to the present invention, may not exceed 200 mg, or 100 mg. For example, for a single dose, the amount of the antibody in the pharmaceutical composition according to the present invention, may not exceed 50 mg.

Pharmaceutical compositions typically include an “effective” amount of one or more antibodies of the invention, i.e. an amount that is sufficient to treat, ameliorate, attenuate, reduce or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. For purposes of the present invention, an effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody of the present invention (e.g. amount of the antibody in the pharmaceutical composition) in relation to the bodyweight (e.g., in kg) of the individual to which it is administered.

Moreover, the pharmaceutical composition according to the present invention may also comprise an additional active component, which may be a further antibody or a component, which is not an antibody. Accordingly, the pharmaceutical composition according to the present invention may comprise one or more of the additional active components. In some embodiments, the pharmaceutical composition comprises two distinct antibodies or antigen-binding fragments, in particular two distinct antibodies or antigen-binding fragments (specifically) binding to tetanus toxin. In some embodiments, the each of the two distinct antibodies, or antigen-binding fragments thereof, is an antibody according to the present invention as described above.

The antibody according to the present invention can be present either in the same pharmaceutical composition as the additional active component (e.g., a second antibody as described above) or, alternatively, the antibody according to the present invention is comprised in a first pharmaceutical composition and the additional active component (e.g., a second antibody as described above) is comprised in a second pharmaceutical composition different from the first pharmaceutical composition. Accordingly, if more than one additional active component is envisaged, each additional active component (e.g., a second antibody as described above) and the antibody according to the present invention may be comprised in a different pharmaceutical composition. Such different pharmaceutical compositions may be administered either combined/simultaneously or at separate times or at separate locations (e.g. separate parts of the body).

Accordingly, the present invention also provides a combination of two distinct antibodies, or antigen-binding fragments thereof, wherein each of the two distinct antibodies, or antigen-binding fragments thereof, is an antibody according to the present invention as described above, preferably for use in medicine as described in more detail below.

Furthermore, the present invention also provides a kit of parts comprising two distinct antibodies, or antigen-binding fragments thereof, wherein each of the two distinct antibodies, or antigen-binding fragments thereof, is an antibody according to the present invention as described above. In the kit of parts, the two distinct antibodies may be provided in distinct vessels (e.g., in distinct pharmaceutical compositions).

In particular, for the above-mentioned pharmaceutical composition, combination and kit of parts, the first of the two distinct antibodies, or antigen-binding fragments thereof, of the invention may comprise a heavy chain CDR1 sequence according to SEQ ID NO: 1, a heavy chain CDR2 sequence according to SEQ ID NO: 2, a heavy chain CDR3 sequence according to SEQ ID NO: 3, a light chain CDR1 sequence according to SEQ ID NO: 4, a light chain CDR2 sequence according to SEQ ID NO: 5 or 6, and a light chain CDR3 sequence according to SEQ ID NO: 7; and the second antibody, or antigen-binding fragment thereof, may comprise a heavy chain CDR1 sequence according to SEQ ID NO: 12, a heavy chain CDR2 sequence according to SEQ ID NO: 13, a heavy chain CDR3 sequence according to SEQ ID NO: 14, a light chain CDR1 sequence according to SEQ ID NO: 15, a light chain CDR2 sequence according to SEQ ID NO: 16 or 17, and a light chain CDR3 sequence according to SEQ ID NO: 18.

In some embodiments, the first antibody, or antigen-binding fragment, may comprise a VH sequence according to SEQ ID NO: 8, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity; and a VL sequence according to SEQ ID NO: 10, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5 or 6, and SEQ ID NO: 7, respectively) may be maintained. The second antibody, or antigen-binding fragment, may then comprise a VH sequence according to SEQ ID NO: 19, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity; and a VL sequence according to SEQ ID NO: 20, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity. Thereby, the CDR sequences as defined above (heavy chain CDR1, CDR2, and CDR3 sequences as set forth in SEQ ID NO: 12, SEQ ID NO: 13, and SEQ ID NO: 14, respectively; and light chain CDR1, CDR2, and CDR3 sequences as set forth in SEQ ID NO: 15, SEQ ID NO: 16 or 17, and SEQ ID NO: 18, respectively) may be maintained.

The antibody according to the present invention and the additional active component (e.g., a second antibody as described above) may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In some embodiments, the pharmaceutical composition according to the present invention may not comprise an additional active component (in addition to the antibody of the invention or respective nucleic acids, vectors or cells as described above).

In some embodiments, a composition of the invention may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition of the invention, the antibodies may be in purified form.

The present invention also provides a method of preparing a pharmaceutical composition comprising the steps of: (i) preparing an antibody of the invention; and (ii) admixing the purified antibody with one or more pharmaceutically acceptable excipients, diluents or carriers.

In other embodiments, a method of preparing a pharmaceutical composition comprises the step of: admixing an antibody with one or more pharmaceutically-acceptable carriers, wherein the antibody is a monoclonal antibody that was obtained from a transformed B cell or a cultured plasma cell of the invention.

As an alternative to delivering antibodies or B cells for therapeutic purposes, it is possible to deliver nucleic acid (typically DNA) that encodes the monoclonal antibody of interest derived from the B cell or the cultured plasma cells to a subject, such that the nucleic acid can be expressed in the subject in situ to provide a desired therapeutic effect. Suitable gene therapy and nucleic acid delivery vectors are known in the art.

Pharmaceutical compositions may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions of the invention may also comprise one or more immunoregulatory agents. In some embodiments, one or more of the immunoregulatory agents include(s) an adjuvant.

### Medical Treatments and Other Uses

In a further aspect, the present invention provides the use of the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the pharmaceutical composition according to the present invention, the combination according to the present invention or the kit of parts according to the present invention as a medicament. In particular, the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the pharmaceutical composition according to the present invention, the combination according to the present invention or the kit of parts according to the present invention may be used in prophylaxis and/or treatment of an infection with Clostridium tetani or tetanus.

Accordingly, the present invention also provides a method of ameliorating or reducing infection with Clostridium tetani or tetanus, or lowering the risk of infection with Clostridium tetani or tetanus, comprising: administering to a subject in need thereof, a therapeutically effective amount of the antibody, or an antigen-binding fragment thereof, according to the present invention, the nucleic acid molecule (or the combination of nucleic acid molecules) according to the present invention, the vector (or the combination of vectors) according to the present invention, the cell according to the present invention or the pharmaceutical composition according to the present invention. Moreover, the present invention also provides the use of the antibody according to the present invention, or an antigen-binding fragment thereof, the nucleic acid molecule (or the combination of nucleic acid molecules) according to the present invention, the vector (or the combination of vectors) according to the present invention, the cell according to the present invention or the pharmaceutical composition according to the present invention in the manufacture of a medicament for prophylaxis, treatment or attenuation of infection with Clostridium tetani or tetanus.

Prophylaxis of infection with Clostridium tetani or tetanus refers in particular to prophylactic settings, wherein the subject was not diagnosed for infection with Clostridium tetani or tetanus (either no diagnosis was performed or diagnosis results were negative) and/or the subject does not show symptoms of infection with Clostridium tetani or tetanus. In therapeutic settings, in contrast, the subject is typically diagnosed with infection with Clostridium tetani or tetanus and/or showing symptoms of infection with Clostridium tetani or tetanus. Of note, the terms “treatment” and “therapy”/“therapeutic” of infection with Clostridium tetani or tetanus include (complete) cure as well as attenuation/reduction of infection with Clostridium tetani or tetanus and/or related symptoms.

In some embodiments the subject may be a human. One way of checking efficacy of therapeutic treatment involves monitoring disease symptoms after administration of the composition of the invention. Treatment can be a single dose schedule or a multiple dose schedule. In one embodiment, an antibody, antibody fragment, nucleic acid, vector, cell or composition according to the invention is administered to a subject in need of such treatment. Such a subject includes, but is not limited to, one who is particularly at risk of or susceptible to infection with Clostridium tetani or tetanus, including, for example, an immunocompromised subject.

Antibodies and fragments thereof as described in the present invention may also be used for the diagnosis of infection with Clostridium tetani or tetanus. Methods of diagnosis may include contacting an antibody with a sample. Such samples may be isolated from a subject, for example an isolated tissue sample, for example taken from nasal passages, sinus cavities, salivary glands, lung, liver, pancreas, kidney, ear, eye, placenta, alimentary tract, heart, ovaries, pituitary, adrenals, thyroid, brain, skin or blood, such as plasma or serum. For example, the antibody, or an antigen-binding fragment thereof, may be contacted with an (isolated) blood sample (e.g., whole blood, plasma or serum). The methods of diagnosis may also include the detection of an antigen/antibody complex, in particular following the contacting of an antibody with a sample. Such a detection step is typically performed at the bench, i.e. without any contact to the human or animal body. Examples of detection methods are well-known to the person skilled in the art and include, e.g., ELISA (enzyme-linked immunosorbent assay). Accordingly, the diagnosis may be performed in vitro, for example by using an isolated sample as described above (and an in vitro detection step of an antigen/antibody complex). Accordingly, the antibody, or an antigen-binding fragment thereof, may be used in (in vitro) diagnosis of infection with Clostridium tetani or tetanus.

Accordingly, the antibody of the present invention, or an antigen-binding fragment thereof, may be used in an (in vitro) method for detecting the antigen, namely, tetanus toxin. Likewise, the antibody of the present invention, or an antigen-binding fragment thereof, may be used in an (in vitro) method for binding tetanus toxin target protein/antigen. For detecting tetanus toxin (antigen), the antibody may be brought in contact with a (isolated) sample (i.e., a sample to be tested for the presence of the antigen). By the specific binding of the antibody to its antigen (tetanus toxin), an antibody/antigen complex is formed, which can be easily detected by methods known in the art.

Such a detection method may be used in the context of (in vitro) diagnosis (with samples isolated from a human or animal body), but also for testing other (e.g., production/manufacture) samples, such as vaccine samples. Accordingly, antibodies, antibody fragment, or variants thereof, as described in the present invention may also be used in a non-therapeutic/non-diagnostic context, e.g. in a vaccine development or manufacture. The present invention therefore also provides the use of the antibody of the present invention, or an antigen-binding fragment thereof, for testing vaccines, in particular whether the antigen (i.e., the desired antigen contained in the vaccine, such as tetanus toxin) is properly generated and/or folded (and/or in the correct conformation). Accordingly, the antibodies may be used for monitoring vaccine manufacture with the desired immunogenicity. To this end, the antibody may be brought in contact with the vaccine, e.g. as described above. Furthermore, the present invention also encompasses the use of the antibody of the present invention, or an antigen-binding fragment thereof, for monitoring the quality of anti-tetanus vaccines by checking whether the vaccine contains the desired antigen, e.g. tetanus toxin, or a fragment or variant thereof. More specifically, the antibody may be used to check the conformation of the antigen, or an epitope thereof, in a vaccine. Also modified versions of the antigen (tetanus toxin) can be tested with the antibodies of the invention, such as fragments and variants of tetanus toxin, which may be useful in a vaccine.

### BRIEF DESCRIPTION OF THE FIGURES

In the following a brief description of the appended FIGURES will be given. The figures are intended to illustrate the present invention in more detail. However, they are not intended to limit the subject matter of the invention in any way.

FIG. 1 shows for Example 1 the binding properties of monoclonal antibodies isolated from donors vaccinated with tetanus toxoid (TT) to TT.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying FIGURES and the examples below. All such modifications fall within the scope of the appended claims.

### Example 1: Identification and Characterization of Human Monoclonal Antibodies TT104 and TT110

Peripheral blood samples were collected from donors who underwent routine vaccinations with tetanus toxoid (TT). Memory B cells were isolated through magnetic cell sorting with anti-CD19-PECy7 antibodies and mouse anti-PE microbeads followed by FACS sorting using Alexa Fluor 647-conjugated goat anti-human IgG, Alexa Fluor 647-conjugated goat anti-human IgM and PE-labeled anti-human IgD. Sorted IgG memory B cells were immortalized with Epstein-Barr virus (EBV) and plated in single-cell cultures in the presence of CpG-DNA and irradiated PBMC-feeder cells cells (as described in Traggiai E. et al., 2004, Nat Med 10(8): 871-5 and WO2004/076677).

Two weeks post-immortalization, the culture supernatants were tested for binding to TT by ELISA. Briefly, ELISA plates were coated with 1 μg/ml of recombinant TT. Plates were blocked with 1% BSA and incubated with titrated antibodies, followed by 1/500 alkaline phosphatase (AP)-conjugated goat anti-human IgG. Plates were then washed, substrate (para-nitrophenyl phosphate (p-NPP), Sigma) was added and plates were read at 405 nm.

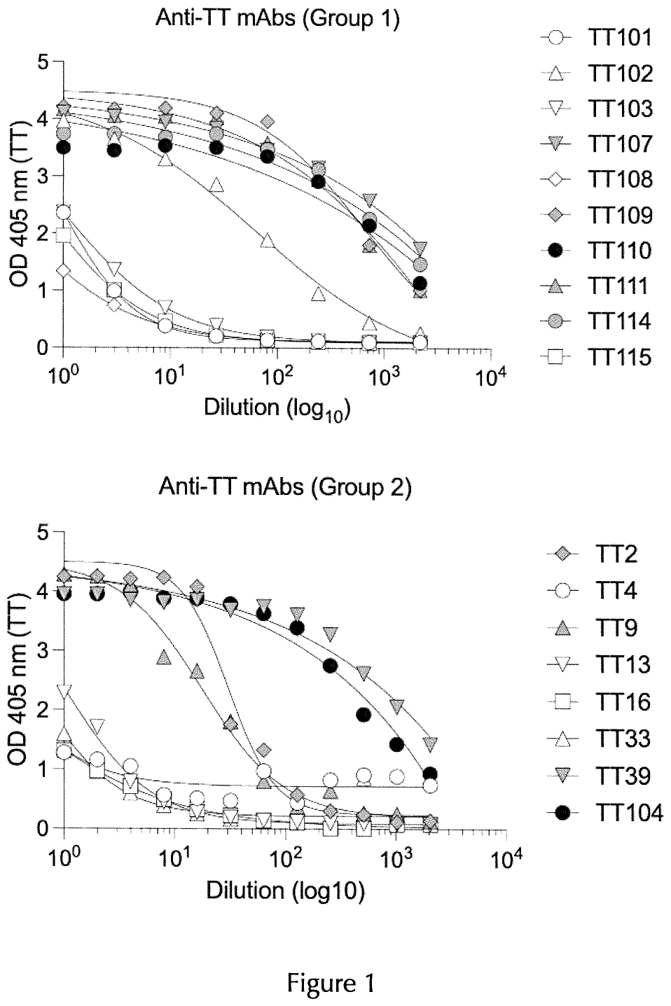

Results are shown in FIG. 1 and Table 3. These data show that several immortalized B cell clones that produced TT-specific monoclonal antibodies were identified; the antibody V genes of 10 TT-specific monoclonal antibodies were sequenced and analyzed using the IMGT database (IMGT: http://www.imgt.org/; cf. Lefranc, M.-P. et al. (2009) Nucleic Acids Res. 37, D1006-D1012).

TABLE 3
EC50 values (Dilution factor, DF) and VDJ-gene (Heavy chain)
and VJ-gene (Light chain) usage of TT-specific monoclonal
antibodies isolated from TT-vaccinated donors.

| mAbs | EC50 | Heavy chain | | | Light chain | |
|---|---|---|---|---|---|---|
| (Group 1) | (DF) | VDJ genes | | | VJ genes | |
| TT101 | 1.03E+00 | NA | | | NA | |
| TT102 | 6.40E+01 | VH3-48*02 | JH4*02 | D5-18*01 | VK1-33*01 | JK2*01 |
| TT103 | 1.06E+00 | VH3-23*04 | JH3*01 | D3-10*01 | VK3-20*01 | JK1*01 |
| TT107 | 7.86E+15 | VH3-11*06 | JH4*02 | D2-15*01 | VK3-20*01 | JK1*01 |
| TT108 | 3.11E−01 | NA | | | NA | |
| TT109 | 6.80E+02 | VH3-30*03 | JH5*01 | D5-18*01 | VK2-30*01 | JK1*01 |
| TT110 | 8.41E+19 | VH1-46*02 | JH4*02 | D5-18*01 | VK3-15*01 | JK1*01 |
| TT111 | 6.66E+03 | NA | | | NA | |
| TT114 | 4.11E+17 | VH4-34*01 | JH5*02 | D3-10*01 | VK1-27*01 | JK4*01 |
| TT115 | 7.20E−01 | NA | | | NA | |
| mAbs | EC50 | | | | | |
| (Group 2) | (DF) | | | | | |
| TT2 | 3.06E+01 | NA | | | NA | |
| TT4 | 2.37E−01 | NA | | | NA | |
| TT9 | 1.79E+01 | NA | | | NA | |
| TT13 | 1.06E+00 | VH3-30-3*01 | JH2*01 | D2-15*01 | VL9-49*03 | JL3*02 |
| TT16 | 2.34E−01 | NA | | | NA | |
| TT33 | 5.43E−01 | VH3-15*01 | JH4*02 | D1-26*01 | VL2-23*01 | JL1*01 |
| TT39 | 7.25E+14 | VH2-5*02 | JH4*02 | D6-19*01 | VL2-14*01 | JL2*01 |
| TT104 | 7.72E+10 | VH4-39*03 | JH5*02 | D1-1*01 | VK3-15*01 | JK2*01 |

NA, not available

Among the antibodies with the highest binding affinities, TT110 and TT104 were selected and FABs of these antibodies were produced.

TABLE OF SEQUENCES AND SEQ ID NUMBERS (SEQUENCE LISTING):

| | | |
|---|---|---|
| SEQ ID NO | Sequence | Remarks |

Amino acid sequences
TT104

| | | |
|---|---|---|
| SEQ ID NO: 1 | DDSMGSSTYY | CDRH1 |
| SEQ ID NO: 2 | VYYNGVT | CDRH2 |
| SEQ ID NO: 3 | ARQADNWFDP | CDRH3 |
| SEQ ID NO: 4 | QSVGSD | CDRL1 |
| SEQ ID NO: 5 | GAS | CDRL2 |
| SEQ ID NO: 6 | LIYGASYRA | CDRL2 long |
| SEQ ID NO: 7 | QQYYNWPPYT | CDRL3 |
| SEQ ID NO: 8 | QLQLQESGPGLVRPSETLSLTCSVSDDSMGSSTY | VH |
| | YWAWIRQPPGKGLEWIGNVYYNGVTYYNSSLESR | |
| | LTLSIDPSKNQFSLILSSVTAADTAVYYCARQAD | |
| | NWFDPWGQGTLVTVSS | |
| SEQ ID NO: 9 | EIMMTQSPATLSVSPGERATLSCRASQSVGSDLA | VL |
| | WYQQKPGQAPRLLIYGASYRATGIPARFSGGGSG | |
| | TEFTLTISSMQSEDFAVYYCQQYYNWPPYTFGQG | |
| | TKVEIK | |
| SEQ ID NO: 10 | QLQLQESGPGLVRPSETLSLTCSVSDDSMGSSTY | Fab heavy chain |
| | YWAWIRQPPGKGLEWIGNVYYNGVTYYNSSLESR | |
| | LTLSIDPSKNQFSLILSSVTAADTAVYYCARQAD | |
| | NWFDPWGQGTLVTVSSASTKGPSVFPLAPSSKST | |
| | SGGTAALGCLVKDYFPEPVTVSWNSGALTSGVHT | |
| | FPAVLQSSGLYSLSSVVTVPSSSLGTQTYICNVN | |
| | HKPSNTKVDKRVEPKSC | |
| SEQ ID NO: 11 | EIMMTQSPATLSVSPGERATLSCRASQSVGSDLA | Fab light chain |
| | WYQQKPGQAPRLLIYGASYRATGIPARFSGGGSG | |
| | TEFTLTISSMQSEDFAVYYCQQYYNWPPYTFGQG | |
| | TKVEIKRTVAAPSVFIFPPSDEQLKSGTASVVCL | |
| | LNNFYPREAKVQWKVDNALQSGNSQESVTEQDSK | |
| | DSTYSLSSTLTLSKADYEKHKVYACEVTHQGLSS | |
| | PVTKSFNRGEC | |

TT110

| | | |
|---|---|---|
| SEQ ID NO: 12 | GFTLNNYY | CDRH1 |
| SEQ ID NO: 13 | FNPSSGTR | CDRH2 |
| SEQ ID NO: 14 | ARIGGSTYGRLMTYYFDH | CDRH3 |
| SEQ ID NO: 15 | RSISTT | CDRL1 |
| SEQ ID NO: 16 | GAS | CDRL2 |
| SEQ ID NO: 17 | LIYGASTRA | CDRL2 long |
| SEQ ID NO: 18 | QQYNDWPVT | CDRL3 |
| SEQ ID NO: 19 | QVQLMQSGAEVQKPGASVKVSCQASGFTLNNYYI | VH |
| | HWLRQAPGQGFEWMGIFNPSSGTRTYAQKFQGRI | |
| | SMTADASTTTLYMELSGLTSEDAGVYFCARIGGS | |
| | TYGRLMTYYFDHWGQGTVVAVSS | |
| SEQ ID NO: 20 | EVVLTQSPVTLSVSPGGRGTLSCRASRSISTTLA | VL |
| | WYQQKPGQAPRLLIYGASTRATGIPARFTGSGSG | |
| | TEFTLTISSLQSEDFAVYYCQQYNDWPVTFGQGT | |
| | QVEVK | |
| SEQ ID NO: 21 | QVQLMQSGAEVQKPGASVKVSCQASGFTLNNYYI | Fab heavy chain |
| | HWLRQAPGQGFEWMGIFNPSSGTRTYAQKFQGRI | |
| | SMTADASTTTLYMELSGLTSEDAGVYFCARIGGS | |
| | TYGRLMTYYFDHWGQGTVVAVSSASTKGPSVFPL | |
| | APSSKSTSGGTAALGCLVKDYFPEPVTVSWNSGA | |
| | LTSGVHTFPAVLQSSGLYSLSSVVTVPSSSLGTQ | |
| | TYICNVNHKPSNTKVDKRVEPKSC | |
| SEQ ID NO: 22 | EVVLTQSPVTLSVSPGGRGTLSCRASRSISTTLA | Fab light chain |
| | WYQQKPGQAPRLLIYGASTRATGIPARFTGSGSG | |
| | TEFTLTISSLQSEDFAVYYCQQYNDWPVTFGQGT | |
| | QVEVKRTVAAPSVFIFPPSDEQLKSGTASVVCLL | |
| | NNFYPREAKVQWKVDNALQSGNSQESVTEQDSKD | |
| | STYSLSSTLTLSKADYEKHKVYACEVTHQGLSSP | |
| | VTKSFNRGEC | |

Constant regions

| | | |
|---|---|---|
| SEQ ID NO: 23 | ASTKGPSVFPLAPSSKSTSGGTAALGCLVKDYFP | IgG heavy chain |
| | EPVTVSWNSGALTSGVHTFPAVLQSSGLYSLSSV | |
| | VTVPSSSLGTQTYICNVNHKPSNTKVDKKVEPKS | |
| | CDKTHTCPPCPAPELLGGPSVFLFPPKPKDTLMI | |
| | SRTPEVTCVVVDVSHEDPEVKFNWYVDGVEVHNA | |
| | KTKPREEQYNSTYRVVSVLTVLHQDWLNGKEYKC | |
| | KVSNKALPAPIEKTISKAKGQPREPQVYTLPPSR | |
| | DELTKNQVSLTCLVKGFYPSDIAVEWESNGQPEN | |
| | NYKTTPPVLDSDGSFFLYSKLTVDKSRWQQGNVF | |
| | SCSVLHEALHSHYTQKSLSLSPGK | |
| SEQ ID NO: 24 | GQPKAAPSVTLFPPSSEELQANKATLVCLISDFY | gG lambda light |
| | PGAVTVAWKADSSPVKAGVETTTPSKQSNNKYAA | chain |
| | SSYLSLTPEQWKSHRSYSCQVTHEGSTVEKTVAP | |
| | TECS | |
| SEQ ID NO: 25 | RTVAAPSVFIFPPSDEQLKSGTASVVCLLNNFYP | IgG kappa light |
| | REAKVQWKVDNALQSGNSQESVTEQDSKDSTYSL | chain |
| | SSTLTLSKADYEKHKVYACEVTHQGLSSPVTKSF | |
| | NRGEC | |
| SEQ ID NO: 26 | ASTKGPSVFPLAPSSKSTSGGTAALGCLVKDYFP | Fab heavy chain |
| | EPVTVSWNSGALTSGVHTFPAVLQSSGLYSLSSV | |
| | VTVPSSSLGTQTYICNVNHKPSNTKVDKRVEPKS | |
| | C | |

Nucleic acid sequences
TT104

| | | |
|---|---|---|
| SEQ ID NO: 27 | gatgactccatgggcagcagtacttactac | CDRH1 |
| SEQ ID NO: 28 | gtctattataatggagtcacc | CDRH2 |
| SEQ ID NO: 29 | gcgagacaagcagacaactggttcgacccc | CDRH3 |
| SEQ ID NO: 30 | cagagtgttggcagcgac | CDRL1 |
| SEQ ID NO: 31 | ggagcatcc | CDRL2 |
| SEQ ID NO: 32 | ctcatctatggagcatcctacagggcc | CDRL2 long |
| SEQ ID NO: 33 | cagcagtattacaactggcccccgtacact | CDRL3 |
| SEQ ID NO: 34 | cagctgcagctgcaggagtcgggcccaggactgg | VH |
| | tgaggccctcggagaccctgtccctcacctgcag | |
| | tgtctctgatgactccatgggcagcagtacttac | |
| | tactgggcctggatccgccagcccccagggaagg | |
| | gactggagtggattgggaatgtctattataatgg | |
| | agtcacctactacaactcgtccctcgagagtcga | |
| | ctcaccttatccatcgacccgtctaagaaccagt | |
| | tctccctgatcttgagttctgtgaccgccgcaga | |
| | cacggccgtatattactgtgcgagacaagcagac | |
| | aactggttcgacccctggggccagggaaccctgg | |
| | tcaccgtctcgtca | |
| SEQ ID NO: 35 | gaaataatgatgacgcagtctccagccaccctgt | VL |
| | ctgtgtctccaggggaaagagccaccctctcctg | |
| | cagggccagtcagagtgttggcagcgacttggct | |
| | tggtaccaacagaaacctggccaggctcccaggc | |
| | tcctcatctatggagcatcctacagggccactgg | |
| | tatcccagccaggttcagtggcggtgggtctggg | |
| | acagagttcactctcaccatcagcagtatgcagt | |
| | ctgaagattttgcagtttattactgtcagcagta | |
| | ttacaactggcccccgtacacttttggccagggg | |
| | accaaggtggagatcaaa | |
| SEQ ID NO: 36 | cagctgcagctgcaggagtctggaccaggactgg | Fab heavy chain |
| | tgcggccctccgagaccctgtctctgacatgctc | |
| | tgtgagcgacgattctatgggcagctccacctac | |
| | tattgggcctggatcagacagccccctggcaagg | |
| | gactggagtggatcggcaacgtgtactataatgg | |
| | cgtgacctactataactctagcctggagtctagg | |
| | ctgacactgagcatcgacccttccaagaatcagt | |
| | tcagcctgatcctgtcctctgtgaccgccgccga | |
| | tacagccgtgtactattgtgcccggcaggccgac | |
| | aactggtttgatccttggggacagggcaccctgg | |
| | tgacagtgagctccgcgtcgaccaagggcccatc | |
| | ggtcttccccctggcaccctcctccaagagcacc | |
| | tctgggggcacagcggccctgggctgcctggtca | |
| | aggactacttccccgaacctgtgacggtctcgtg | |
| | gaactcaggcgccctgaccagcggcgtgcacacc | |
| | ttcccggctgtcctacagtcctcaggactctact | |
| | ccctcagcagcgtggtgaccgtgccctccagcag | |
| | cttgggcacccagacctacatctgcaacgtgaat | |
| | cacaagcccagcaacaccaaggtggacaagagag | |
| | ttgagcccaaatcttgttag | |
| SEQ ID NO: 37 | gaaataatgatgacgcagtctccagccaccctgt | Fab light chain |
| | ctgtgtctccaggggaaagagccaccctctcctg | |
| | cagggccagtcagagtgttggcagcgacttggct | |
| | tggtaccaacagaaacctggccaggctcccaggc | |
| | tcctcatctatggagcatcctacagggccactgg | |
| | tatcccagccaggttcagtggcggtgggtctggg | |
| | acagagttcactctcaccatcagcagtatgcagt | |
| | ctgaagattttgcagtttattactgtcagcagta | |
| | ttacaactggcccccgtacacttttggccagggg | |
| | accaaggtggagatcaaacgtacggtggctgcac | |
| | catctgtcttcatcttcccgccatctgatgagca | |
| | gttgaaatctggaactgcctctgttgtgtgcctg | |
| | ctgaataacttctatcccagagaggccaaagtac | |
| | agtggaaggtggataacgccctccaatcgggtaa | |
| | ctcccaggagagtgtcacagagcaggacagcaag | |
| | gacagcacctacagcctcagcagcaccctgacgc | |
| | tgagcaaagcagactacgagaaacacaaagtcta | |
| | cgcctgcgaagtcacccatcagggcctgagctcg | |
| | cccgtcacaaagagcttcaacaggggagagtgtt | |
| | ag | |

TT110

| | | |
|---|---|---|
| SEQ ID NO: 38 | ggattcaccctcaacaattactat | CDRH1 |
| SEQ ID NO: 39 | ttcaaccctagtagtgggaccaga | CDRH2 |
| SEQ ID NO: 40 | gcgagaattggaggctccacttatggccgactaa | CDRH3 |
| | tgacctactacttt | |
| | gatcat | |
| SEQ ID NO: 41 | cggagtattagcaccacc | CDRL1 |
| SEQ ID NO: 42 | ggtgcttcc | CDRL2 |
| SEQ ID NO: 43 | ctcatttatggtgcttccaccagggcc | CDRL2 long |
| SEQ ID NO: 44 | cagcagtataatgactggcccgtgacg | CDRL3 |
| SEQ ID NO: 45 | caggttcagctaatgcagtctggggctgaggtgc | VH |
| | agaagcctggggcctcagtgaaagtgtcctgcca | |
| | ggcatctggattcaccctcaacaattactatatt | |
| | cactggctacgacaggcccctggacaagggtttg | |
| | agtggatgggaattttcaaccctagtagtgggac | |
| | cagaacttacgcgcagaagttccagggcagaatc | |
| | agtatgaccgccgacgcgtcaacgacgacactct | |
| | acatggagctgagcggcctgacttcggaagacgc | |
| | gggcgtatatttctgtgcgagaattggaggctcc | |
| | acttatggccgactaatgacctactactttgatc | |
| | attggggccagggaacggtggtcgccgtctcctc | |
| | a | |
| SEQ ID NO: 46 | gaggtagtgttgacacagtctccggtcaccctgt | VL |
| | ctgtgtctccaggggggagaggcaccctctcctg | |
| | cagggccagtcggagtattagcaccaccttagcc | |
| | tggtaccagcagaaacctggccaggctcccaggc | |
| | tcctcatttatggtgcttccaccagggccactgg | |
| | tatcccagccaggttcactggcagtgggtctggg | |
| | acagagttcacgctcaccatcagcagcctacagt | |
| | ctgaagatttcgcagtttattattgtcagcagta | |
| | taatgactggcccgtgacgttcggccaggggacc | |
| | caggtggaagtcaag | |
| SEQ ID NO: 47 | caggttcagctaatgcagtctggggctgaggtgc | Fab heavy chain |
| | agaagcctggggcctcagtgaaagtgtcctgcca | |
| | ggcatctggattcaccctcaacaattactatatt | |
| | cactggctacgacaggcccctggacaagggtttg | |
| | agtggatgggaattttcaaccctagtagtgggac | |
| | cagaacttacgcgcagaagttccagggcagaatc | |
| | agtatgaccgccgacgcgtcaacgacgacactct | |
| | acatggagctgagcggcctgacttcggaagacgc | |
| | gggcgtatatttctgtgcgagaattggaggctcc | |
| | acttatggccgactaatgacctactactttgatc | |
| | attggggccagggaacggtggtcgccgtctcctc | |
| | agcgtcgaccaagggcccatcggtcttccccctg | |
| | gcaccctcctccaagagcacctctgggggcacag | |
| | cggccctgggctgcctggtcaaggactacttccc | |
| | cgaacctgtgacggtctcgtggaactcaggcgcc | |
| | ctgaccagcggcgtgcacaccttcccggctgtcc | |
| | tacagtcctcaggactctactccctcagcagcgt | |
| | ggtgaccgtgccctccagcagcttgggcacccag | |
| | acctacatctgcaacgtgaatcacaagcccagca | |
| | acaccaaggtggacaagagagttgagcccaaatc | |
| | ttgttag | |
| SEQ ID NO: 48 | gaggtagtgttgacacagtctccggtcaccctgt | Fab light chain |
| | ctgtgtctccaggggggagaggcaccctctcctg | |
| | cagggccagtcggagtattagcaccaccttagcc | |
| | tggtaccagcagaaacctggccaggctcccaggc | |
| | tcctcatttatggtgcttccaccagggccactgg | |
| | tatcccagccaggttcactggcagtgggtctggg | |
| | acagagttcacgctcaccatcagcagcctacagt | |
| | ctgaagatttcgcagtttattattgtcagcagta | |
| | taatgactggcccgtgacgttcggccaggggacc | |
| | caggtggaagtcaagcgtacggtggctgcaccat | |
| | ctgtcttcatcttcccgccatctgatgagcagtt | |
| | gaaatctggaactgcctctgttgtgtgcctgctg | |
| | aataacttctatcccagagaggccaaagtacagt | |
| | ggaaggtggataacgccctccaatcgggtaactc | |
| | ccaggagagtgtcacagagcaggacagcaaggac | |
| | agcacctacagcctcagcagcaccctgacgctga | |
| | gcaaagcagactacgagaaacacaaagtctacgc | |
| | ctgcgaagtcacccatcagggcctgagctcgccc | |
| | gtcacaaagagcttcaacaggggagagtgttag | |

1. An antibody, or an antigen-binding fragment thereof, which binds to tetanus toxin.

2. The antibody, or an antigen-binding fragment thereof, according to claim 1, wherein the antibody or the antigen-binding fragment thereof comprises (i) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 17, and SEQ ID NO: 18, respectively.

3. The antibody, or an antigen-binding fragment thereof, according to any one of the previous claims, wherein the antibody or the antigen-binding fragment thereof comprises (i) heavy chain CDR1, CDR2, and CDR3 sequences having at least 80% sequence identity with the amino acid sequences of SEQ ID NO: 1, SEQ ID NO: 2, and SEQ ID NO: 3, respectively, and light chain CDR1, CDR2, and CDR3 sequences having at least 80% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 80% sequence identity with the amino acid sequences of SEQ ID NO: 1, SEQ ID NO: 2, and SEQ ID NO: 3, respectively, and light chain CDR1, CDR2, and CDR3 sequences having at least 80% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 80% sequence identity with the amino acid sequences of SEQ ID NO: 12, SEQ ID NO: 13, and SEQ ID NO: 14, respectively, and light chain CDR1, CDR2, and CDR3 sequences having at least 80% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 80% sequence identity with the amino acid sequences of SEQ ID NO: 12, SEQ ID NO: 13, and SEQ ID NO: 14, respectively, and light chain CDR1, CDR2, and CDR3 sequences having at least 80% sequence identity with the amino acid sequences of SEQ ID NO: 15, SEQ ID NO: 17, and SEQ ID NO: 18, respectively.

4. The antibody, or an antigen-binding fragment thereof, according to any one of the previous claims, wherein the antibody or the antigen-binding fragment thereof comprises (i) heavy chain CDR1, CDR2, and CDR3 sequences having at least 90% sequence identity with the amino acid sequences of SEQ ID NO: 1, SEQ ID NO: 2, and SEQ ID NO: 3, respectively, and light chain CDR1, CDR2, and CDR3 sequences having at least 90% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 90% sequence identity with the amino acid sequences of SEQ ID NO: 1, SEQ ID NO: 2, and SEQ ID NO: 3, respectively, and light chain CDR1, CDR2, and CDR3 sequences having at least 90% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 90% sequence identity with the amino acid sequences of SEQ ID NO: 12, SEQ ID NO: 13, and SEQ ID NO: 14, respectively, and light chain CDR1, CDR2, and CDR3 sequences having at least 90% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 90% sequence identity with the amino acid sequences of SEQ ID NO: 12, SEQ ID NO: 13, and SEQ ID NO: 14, respectively, and light chain CDR1, CDR2, and CDR3 sequences having at least 90% sequence identity with the amino acid sequences of SEQ ID NO: 15, SEQ ID NO: 17, and SEQ ID NO: 18, respectively.

5. The antibody, or an antigen-binding fragment thereof, according to any one of the previous claims, wherein the antibody or the antigen-binding fragment thereof comprises (i) a heavy chain CDR1 sequence according to SEQ ID NO: 1, a heavy chain CDR2 sequence according to SEQ ID NO: 2, a heavy chain CDR3 sequence according to SEQ ID NO: 3, a light chain CDR1 sequence according to SEQ ID NO: 4, a light chain CDR2 sequence according to SEQ ID NO: 5 or 6, and a light chain CDR3 sequence according to SEQ ID NO: 7; or (ii) a heavy chain CDR1 sequence according to SEQ ID NO: 12, a heavy chain CDR2 sequence according to SEQ ID NO: 13, a heavy chain CDR3 sequence according to SEQ ID NO: 14, a light chain CDR1 sequence according to SEQ ID NO: 15, a light chain CDR2 sequence according to SEQ ID NO: 16 or 17, and a light chain CDR3 sequence according to SEQ ID NO: 18.

6. The antibody, or an antigen-binding fragment thereof, according to any one of the previous claims, wherein the antibody or the antigen-binding fragment thereof comprises (i) a heavy chain variable region comprising an amino acid sequence having at least 70% identity to SEQ ID NO: 8 and a light chain variable region comprising an amino acid sequence having at least 70% identity to SEQ ID NO: 9; or (ii) a heavy chain variable region comprising an amino acid sequence having at least 70% identity to SEQ ID NO: 19 and a light chain variable region comprising an amino acid sequence having at least 70% identity to SEQ ID NO: 20.

7. The antibody, or an antigen-binding fragment thereof, according to any one of the previous claims, wherein the antibody or the antigen-binding fragment thereof comprises (i) a heavy chain variable region comprising an amino acid sequence having at least 75% identity to SEQ ID NO: 8 and a light chain variable region comprising an amino acid sequence having at least 75% identity to SEQ ID NO: 9; or (ii) a heavy chain variable region comprising an amino acid sequence having at least 75% identity to SEQ ID NO: 19 and a light chain variable region comprising an amino acid sequence having at least 75% identity to SEQ ID NO: 20.

8. The antibody, or an antigen-binding fragment thereof, according to any one of the previous claims, wherein the antibody or the antigen-binding fragment thereof comprises (i) a heavy chain variable region comprising an amino acid sequence having at least 80% identity to SEQ ID NO: 8 and a light chain variable region comprising an amino acid sequence having at least 80% identity to SEQ ID NO: 9; or (ii) a heavy chain variable region comprising an amino acid sequence having at least 80% identity to SEQ ID NO: 19 and a light chain variable region comprising an amino acid sequence having at least 80% identity to SEQ ID NO: 20.

9. The antibody, or an antigen-binding fragment thereof, according to any one of the previous claims, wherein the antibody or the antigen-binding fragment thereof comprises (i) a heavy chain variable region comprising an amino acid sequence having at least 85% identity to SEQ ID NO: 8 and a light chain variable region comprising an amino acid sequence having at least 85% identity to SEQ ID NO: 9; or (ii) a heavy chain variable region comprising an amino acid sequence having at least 85% identity to SEQ ID NO: 19 and a light chain variable region comprising an amino acid sequence having at least 85% identity to SEQ ID NO: 20.

10. The antibody, or an antigen-binding fragment thereof, according to any one of the previous claims, wherein the antibody or the antigen-binding fragment thereof comprises (i) a heavy chain variable region comprising an amino acid sequence having at least 90% identity to SEQ ID NO: 8 and a light chain variable region comprising an amino acid sequence having at least 90% identity to SEQ ID NO: 9; or (ii) a heavy chain variable region comprising an amino acid sequence having at least 90% identity to SEQ ID NO: 19 and a light chain variable region comprising an amino acid sequence having at least 90% identity to SEQ ID NO: 20.

11. The antibody, or an antigen-binding fragment thereof, according to any one of the previous claims, wherein the antibody or the antigen-binding fragment thereof comprises (i) a heavy chain variable region comprising an amino acid sequence having at least 95% identity to SEQ ID NO: 8 and a light chain variable region comprising an amino acid sequence having at least 95% identity to SEQ ID NO: 9; or (ii) a heavy chain variable region comprising an amino acid sequence having at least 95% identity to SEQ ID NO: 19 and a light chain variable region comprising an amino acid sequence having at least 95% identity to SEQ ID NO: 20.

12. The antibody, or an antigen-binding fragment thereof, according to any one of the previous claims, wherein the antibody or the antigen-binding fragment thereof comprises (i) a heavy chain variable region comprising an amino acid sequence according to SEQ ID NO: 8 and a light chain variable region comprising an amino acid sequence according to SEQ ID NO: 9; or (ii) a heavy chain variable region comprising an amino acid sequence according to SEQ ID NO: 19 and a light chain variable region comprising an amino acid sequence according to SEQ ID NO: 20.

13. The antibody, or an antigen-binding fragment thereof, according to any one of the previous claims, wherein the antibody or the antigen-binding fragment thereof comprises (i) a heavy chain comprising an amino acid sequence having at least 70% identity to SEQ ID NO: 10 and a light chain comprising an amino acid sequence having at least 70% identity to SEQ ID NO: 11; or (ii) a heavy chain comprising an amino acid sequence having at least 70% identity to SEQ ID NO: 21 and a light chain comprising an amino acid sequence having at least 70% identity to SEQ ID NO: 22.

14. The antibody, or an antigen-binding fragment thereof, according to any one of the previous claims, wherein the antibody or the antigen-binding fragment thereof comprises (i) a heavy chain comprising an amino acid sequence having at least 75% identity to SEQ ID NO: 10 and a light chain comprising an amino acid sequence having at least 75% identity to SEQ ID NO: 11; or (ii) a heavy chain comprising an amino acid sequence having at least 75% identity to SEQ ID NO: 21 and a light chain comprising an amino acid sequence having at least 75% identity to SEQ ID NO: 22.

15. The antibody, or an antigen-binding fragment thereof, according to any one of the previous claims, wherein the antibody or the antigen-binding fragment thereof comprises (i) a heavy chain comprising an amino acid sequence having at least 80% identity to SEQ ID NO: 10 and a light chain comprising an amino acid sequence having at least 80% identity to SEQ ID NO: 11; or (ii) a heavy chain comprising an amino acid sequence having at least 80% identity to SEQ ID NO: 21 and a light chain comprising an amino acid sequence having at least 80% identity to SEQ ID NO: 22.

16. The antibody, or an antigen-binding fragment thereof, according to any one of the previous claims, wherein the antibody or the antigen-binding fragment thereof comprises (i) a heavy chain comprising an amino acid sequence having at least 85% identity to SEQ ID NO: 10 and a light chain comprising an amino acid sequence having at least 85% identity to SEQ ID NO: 11; or (ii) a heavy chain comprising an amino acid sequence having at least 85% identity to SEQ ID NO: 21 and a light chain comprising an amino acid sequence having at least 85% identity to SEQ ID NO: 22.

17. The antibody, or an antigen-binding fragment thereof, according to any one of the previous claims, wherein the antibody or the antigen-binding fragment thereof comprises (i) a heavy chain comprising an amino acid sequence having at least 90% identity to SEQ ID NO: 10 and a light chain comprising an amino acid sequence having at least 90% identity to SEQ ID NO: 11; or (ii) a heavy chain comprising an amino acid sequence having at least 90% identity to SEQ ID NO: 21 and a light chain comprising an amino acid sequence having at least 90% identity to SEQ ID NO: 22.

18. The antibody, or an antigen-binding fragment thereof, according to any one of the previous claims, wherein the antibody or the antigen-binding fragment thereof comprises (i) a heavy chain comprising an amino acid sequence having at least 95% identity to SEQ ID NO: 10 and a light chain comprising an amino acid sequence having at least 95% identity to SEQ ID NO: 11; or (ii) a heavy chain comprising an amino acid sequence having at least 95% identity to SEQ ID NO: 21 and a light chain comprising an amino acid sequence having at least 95% identity to SEQ ID NO: 22.

19. The antibody, or an antigen-binding fragment thereof, according to any one of the previous claims, wherein the antibody or the antigen-binding fragment thereof comprises (i) a heavy chain comprising an amino acid sequence according to SEQ ID NO: 10 and a light chain comprising an amino acid sequence according to SEQ ID NO: 11; or (ii) a heavy chain comprising an amino acid sequence according to SEQ ID NO: 21 and a light chain comprising an amino acid sequence according to SEQ ID NO: 22.

20. The antibody, or an antigen-binding fragment thereof, according to any one of the previous claims, wherein the antibody or the antigen-binding fragment thereof is a human antibody.

21. The antibody, or an antigen-binding fragment thereof, according to any one of the previous claims, wherein the antibody, or an antigen-binding fragment thereof, is a monoclonal antibody.

22. The antibody, or an antigen-binding fragment thereof, according to any one of the previous claims, wherein the antibody, or the antigen-binding fragment thereof, is purified.

23. The antibody, or an antigen-binding fragment thereof, according to any one of the previous claims, wherein the antibody comprises an Fc moiety.

24. The antibody according to any one of the previous claims, wherein the antibody is of the IgG type.

25. The antibody according to claim 24, wherein the antibody is of the IgG1 type.

26. The antibody, or an antigen-binding fragment thereof, according to any one of claims 1 to 22, wherein the antibody, or the antigen-binding fragment thereof, is selected from Fab, Fab′, F(ab′)2, Fv or scFv.

27. The antibody, or an antigen-binding fragment thereof, according to claim 26, wherein the antibody, or the antigen-binding fragment thereof, is a Fab.

28. The antibody, or an antigen-binding fragment thereof, according to any one of claims 1 to 23, wherein the antibody, or the antigen-binding fragment thereof, is a single-chain antibody.

29. The antibody, or an antigen-binding fragment thereof, according to any one of the previous claims for use as a medicament.

30. The antibody, or an antigen-binding fragment thereof, according to any one of the previous claims for use in the prophylaxis or treatment of infection with Clostridium tetani or tetanus.

31. A nucleic acid molecule comprising a polynucleotide encoding the antibody, or an antigen-binding fragment thereof, according to any one of claims 1 to 28.

32. The nucleic acid molecule according to claim 31, wherein the polynucleotide encoding the antibody, or an antigen-binding fragment thereof, is codon-optimized.

33. The nucleic acid molecule according to claim 31 or 32 comprising a nucleic acid sequence as set forth in any one of SEQ ID NOs 27-48; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

34. A combination of a first and a second nucleic acid molecule, wherein the first nucleic acid molecule comprises a polynucleotide encoding the heavy chain of the antibody, or an antigen-binding fragment thereof, of any one of claims 1 to 28; and the second nucleic acid molecule comprises a polynucleotide encoding the corresponding light chain of the same antibody, or the same antigen-binding fragment thereof.

35. The combination of nucleic acid molecules according to claim 34, wherein one or both of the polynucleotides encoding the heavy and/or light chain(s) of the antibody, or an antigen-binding fragment thereof, is/are codon-optimized.

36. The combination of nucleic acid molecules according to claim 34 or 35 comprising a nucleic acid sequence as set forth in any one of SEQ ID NOs 27-48; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

37. A vector comprising the nucleic acid molecule according to any one of claims 31 to 33 or the combination of nucleic acid molecules according to any one of claims 34 to 36.

38. A combination of a first and a second vector, wherein the first vector comprises a first nucleic acid molecule as defined in any one of claims 34 to 36 and the second vector comprises the corresponding second nucleic acid molecule as defined in any one of claims 34 to 36.

39. A cell expressing the antibody, or an antigen-binding fragment thereof, according to any one of claims 1 to 28; or comprising the vector according to claim 37 or 38.

40. A pharmaceutical composition comprising the antibody, or an antigen-binding fragment thereof, according to any one of claims 1 to 28, the nucleic acid according to any one of claims 31 to 33, the combination of nucleic acids according to any one of claims 34 to 36, the vector according to claim 37, the combination of vectors according to claim 38 or the cell according to claim 39, and, optionally, a pharmaceutically acceptable excipient, diluent or carrier.

41. The antibody, or an antigen-binding fragment thereof, according to any one of claims 1 to 28, the nucleic acid according to any one of claims 31 to 33, the combination of nucleic acids according to any one of claims 34 to 36, the vector according to claim 37, the combination of vectors according to claim 38 or the cell according to claim 39, or the pharmaceutical composition according to claim 40 for use as a medicament; optionally in the prophylaxis or treatment of infection with Clostridium tetani or tetanus.

42. Use of the antibody, or an antigen-binding fragment thereof, according to any one of claims 1 to 28 in (in-vitro) diagnosis of infection with Clostridium tetani or tetanus.

43. Use of the antibody, or an antigen-binding fragment thereof, according to any one of claims 1 to 28 for monitoring the quality of an anti-tetanus vaccine by checking the conformation of an antigen, or an epitope thereof, comprised in said vaccine.

44. Use of the antibody, or an antigen-binding fragment thereof, according to any one of claims 1 to 28, the nucleic acid according to any one of claims 31 to 33, the combination of nucleic acids according to any one of claims 34 to 36, the vector according to claim 37, the combination of vectors according to claim 38 or the cell according to claim 39, or the pharmaceutical composition according to claim 40 in the manufacture of a medicament for prophylaxis, treatment or attenuation of infection with Clostridium tetani or tetanus.

45. A method of reducing infection with Clostridium tetani or tetanus, or lowering the risk of infection with Clostridium tetani or tetanus, comprising: administering to a subject in need thereof, a therapeutically effective amount of antibody, or an antigen-binding fragment thereof, according to any one of claims 1 to 28, the nucleic acid according to any one of claims 31 to 33, the combination of nucleic acids according to any one of claims 34 to 36, the vector according to claim 37, the combination of vectors according to claim 38 or the cell according to claim 39, or the pharmaceutical composition according to claim 40.
